(19) United States
(12) Patent Application Publication
(10) Pub. No.: US 2007/0060534 A1
(43) Pub. Date: Mar. 15, 2007

(54) Anthracycline analogs

(75) Inventors: Mark Matteucci, Portola Valley, CA (US); Jian-Xin Duan, South San Francisco, CA (US); Xiaohong Cai, Burlingame, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Threshold Pharmaceuticals, Inc., Redwood City, CA (US)

(21) Appl. No.: 11/478,937

(22) Filed: Jun. 30, 2006

Related U.S. Application Data
(60) Provisional application No. 60/696,356, filed on Jun. 30, 2005.

(51) Int. Cl. A61K 31/704; C07H 15/24

(52) U.S. Cl. 514/34; 536/6.4

(57) ABSTRACT

Anthracycline analogs and their bioconjugates are useful as anticancer agents.

### CROSS-REFERENCE TO RELATED APPLICATION

This application claims benefit of U.S. provisional patent application No. 60/696,356, filed 30 Jun. 2005, which is incorporated herein by reference.

### FIELD OF THE INVENTION

The present invention provides compounds, compositions, and methods useful in the treatment of cancer and other hyperproliferative diseases and relates to the fields of medicine, medicinal chemistry, pharmacology, and chemistry.

### BACKGROUND OF THE INVENTION

Cancer generally refers to one of a group of more than 100 diseases caused by the uncontrolled growth and spread of abnormal cells that can take the form of solid tumors, lymphomas, and non-solid cancers such as leukemia. Unlike normal cells, which reproduce until maturation is attained and then only as necessary for replacement, cancer cells divide uncontrolled, disrupt normal tissue function and kill the normal tissue.

Radiation therapy and surgical procedure are used for treatment of cancer, particularly those that are localized and are in the early to middle stages of cancer. Although there have been great improvements in diagnosis, general patient care, surgical techniques, and local and systemic adjuvant therapies, cancer related mortality is still common.

The spread or progression of cancer from its initial site to other parts of the body is called metastasis and makes cancer fatal. Radiation therapy or surgery can be ineffective for treatment of metastatic cancer particularly in late stage disease. Chemotherapy can be effective at all stages of the disease, but resistance to chemotherapy often leads to treatment failure. Anthracyclines such as doxorubicin, daunorubicin, and the like used in cancer chemotherapy suffer from drug resistance. Resistance to anthracycline drugs occurs via multi-drug resistance where overexpressed p-glycoprotein removes the drugs out of the cancer cell.

Anthracycline analogs are reported in the references, Henry, 1976, Cancer Chemotherapy, ACS Symposium Series, 15-57; Nagy et al.,1996, Proc. Natl. Acad. Sci. USA, 93: 2464-9; Bakina et al., 1999, Anti-Cancer Drug Design, 14: 507-15; Perrin et al., 1999, Nucleic Acids Research, p. 1781; U.S. Pat. Nos. 4,301,277; 4,314,054; 4,464,529; 4,585,859; 4,591,637; 4,826,964; 5,843,903; and U.S. Pat. Nos. 6,184,374; 5,962,216; 5,196,522; 6,218,519; 6,433,150; PCT publication No. WO 98/13059; and Eur. Pat. No. EP 02/90744. There is a need for new treatments for cancer that can preferably overcome cancer resistance mechanisms and preferably for tumor specific delivery. The present invention satisfies that unmet need and provides anthracycline analogs as summarized below.

### BRIEF SUMMARY OF THE INVENTION

In one aspect the present invention provides anthracycline analogs. In one embodiment, the cytotoxicity of an anthracycline analog of the present invention is between 0.1 picoM-1 μM. In another embodiment the cytotoxicity of a compound of the present invention is between 1 picoM to 100 nM.

In another aspect the present invention provides a anthracycline analog-protein bioconjugate. In one embodiment, the protein is an antibody. In another embodiment, the present invention provides a tumor specific anthracycline analog-antibody bioconjugate.

In one aspect the present invention provides a method for synthesizing the anthracycline analogs and the anthracycline analog-protein bioconjugate of the present invention.

In another aspect, the present method provides a pharmaceutical composition or formulation comprising a compound of the present invention and a pharmaceutically acceptable carrier or diluent.

In another aspect, the present invention provides a method of treating cancer or an other hyperproliferative disease comprising administering a therapeutically effective amount of compound of the present invention to a patient or subject in need of such therapy. These and other aspects and embodiments of the present invention are described below in detail.

### DETAILED DESCRIPTION OF THE INVENTION

### 1. DEFINITIONS

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, “a” or “an” means “at least one” or “one or more.”

“Alkyl” refers to a linear saturated monovalent hydrocarbon radical or a branched saturated monovalent hydrocarbon radical having the number of carbon atoms indicated in the prefix. For example, (C₁-C₆)alkyl is meant to include methyl, ethyl, n-propyl, 2-propyl, n-butyl, 2-butyl, tert-butyl, pentyl, and the like. For each of the definitions herein (e.g., alkyl, alkenyl, alkoxy, araalkyloxy), when a prefix is not included to indicate the number of main chain carbon atoms in an alkyl portion, the radical or portion thereof will have six or fewer main chain carbon atoms.

“Alkenyl” refers to a linear monovalent hydrocarbon radical or a branched monovalent hydrocarbon radical having the number of carbon atoms indicated in the prefix and containing at least one double bond, but no more than three double bonds. For example, (C₂-C₆)alkenyl is meant to include, ethenyl, propenyl, 1,3-butadienyl and the like.

“Acyl” means —CO-“alkyl”.

“Aryl” refers to a monovalent monocyclic or bicyclic aromatic hydrocarbon radical of 6 to 10 ring atoms which is substituted independently with one to four substituents, preferably one, two, or three substituents selected from alkyl, cycloalkyl, cycloalkylalkyl, halo, nitro, cyano, hydroxy, alkoxy, amino, acylamino, mono-alkylamino, di-alkylamino, haloalkyl, haloalkoxy, heteroalkyl, COR (where R is hydrogen, alkyl, cycloalkyl, cycloalkyl-alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl) or —(CR′R″)ₙ—CONR^{x}R^{y} (where n is an integer from 0 to 5, R′ and R″ are independently hydrogen or alkyl, and R^{x} and R^{y} are independently selected from hydrogen, alkyl, cycloalkyl, cycloalkylalkyl, phenyl or phenylalkyl). In one embodiment, R^{x} and R^{y} together is cycloalkyl or heterocyclyl. More specifically the term aryl includes, but is not limited to, phenyl, biphenyl, 1-naphthyl, and 2-naphthyl, and the substituted forms thereof.

“Aroyl” mean —CO-“aryl”.

“Cycloalkyl” refers to a monovalent cyclic hydrocarbon radical of three to seven ring carbons. The cycloalkyl group may have one double bond and may also be optionally substituted independently with one, two, or three substituents selected from alkyl, optionally substituted phenyl, or —C(O)R_{z} (where R_{z} is hydrogen, alkyl, haloalkyl, amino, mono-alkylamino, di-alkylamino, hydroxy, alkoxy, or optionally substituted phenyl). More specifically, the term cycloalkyl includes, for example, cyclopropyl, cyclohexyl, cyclohexenyl, phenylcyclohexyl, 4-carboxycyclohexyl, 2-carboxamidocyclohexenyl, 2-dimethylaminocarbonyl-cyclohexyl, and the like.

“Heteroalkyl” means an alkyl radical as defined herein with one, two or three substituents independently selected from cyano, —OR^{w}, —NR^{x}R^{y}, and S(O)ₚR^{z} (where p is an integer from 0 to 2 ), with the understanding that the point of attachment of the heteroalkyl radical is through a carbon atom of the heteroalkyl radical. R^{w} is hydrogen, alkyl, cycloalkyl, cycloalkyl-alkyl, aryl, araalkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. In one embodiment, R^{x} and R^{y} together is cycloalkyl or heterocyclyl. R^{z} is hydrogen (provided that n is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, fluorine, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{x}R^{y}, or S(O)ₚR^{z} portions.

“Heteroacyl” means —CO-—heteroalkyl”.

“Heteroaryl” means a monovalent monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to four substituents, preferably one or two substituents, selected from alkyl, cycloalkyl, cycloalkyl-alkyl, halo, nitro, cyano, hydroxy, alkoxy, amino, acylamino, mono-alkylamino, di-alkylamino, haloalkyl, haloalkoxy, heteroalkyl, —COR (where R is hydrogen, alkyl, phenyl or phenylalkyl, —(CR′R″)ₙ—COOR (where n is an integer from 0 to 5, R′ and R″ are independently hydrogen or alkyl, and R is hydrogen, alkyl, cycloalkyl, cycloalkyl-alkyl, phenyl or phenylalkyl), or —(CR′R″)ₙ—CONR^{x}R^{y} (where n is an integer from 0 to 5, R′ and R″ are independently hydrogen or alkyl, and R^{x} and R^{y} are, independently of each other, hydrogen, alkyl, cycloalkyl, cycloalkyl-alkyl, phenyl or phenylalkyl). In one embodiment, R^{x} and R^{y} together is cycloalkyl or heterocyclyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenly, and heptaaza-indenyl and the derivatives thereof. Unless indicated otherwise, the arrangement of the hetero atoms within the ring may be any arrangement allowed by the bonding characteristics of the constituent ring atoms.

“Heteroaroyl” means —CO-“heteroaryl”.

“Heterocyclyl” or “cycloheteroalkyl” means a saturated or unsaturated non-aromatic cyclic radical of 3 to 8 ring atoms in which one to four ring atoms are heteroatoms selected from O, NR (where R is independently hydrogen or alkyl) or S(O)ₚ (where p is an integer from 0 to 2), the remaining ring atoms being C, where one or two C atoms may optionally be replaced by a carbonyl group. The heterocyclyl ring may be optionally substituted independently with one, two, or three substituents selected from alkyl, aryl, arylalkyl, heteroaryl, heteroarylalkyl, cycloalkyl, cycloalkylalkyl, halo, nitro, cyano, hydroxy, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically the term heterocyclyl includes, but is not limited to, pyridyl, tetrahydropyranyl, N-methylpiperidin-3-yl, N-methylpyrrolidin-3-yl, 2-pyrrolidon-1-yl, furyl, quinolyl, thienyl, benzothienyl, pyrrolidinyl, piperidinyl, morpholinyl, pyrrolidinyl, tetrahydrofuranyl, tetrahydrothiofuranyl, 1,1-dioxo-hexahydro-1□⁶-thiopyran-4-yl, tetrahydroimidazo [4,5-c] pyridinyl, imidazolinyl, piperazinyl, and piperidin-2-onyl and the derivatives thereof. The prefix indicating the number of carbon atoms (e.g., C₃-C₁₀) refers to the total number of carbon atoms in the portion of the cycloheteroalkyl or heterocyclyl group exclusive of the number of heteroatoms. Each of the groups from “alkyl” to “heterocyclyl” as defined above may be further substituted with substituents, including for example, hydroxy, amino, mono or di(C₁-C₆)alkyl amino, halogen, C₂-C₆ alkenyl ether, cyano, nitro, ethenyl, ethynyl, C₁-C₆ alkoxy, C₁-C₆ alkylthio, —COOH, —CONH₂, mono- or di-(C₁-C₆)alkyl-carboxamido, —SO₂NH₂, —OSO₂—(C₁-C₆)alkyl, mono or di(C₁-C₆)alkylsulfon-amido, aryl, and heteroaryl. A combination of substituents or variables is permissible only if such a combination results in a stable or chemically feasible compound. A stable compound or chemically feasible compound is one in which the chemical structure is not substantially altered when kept at a temperature of 4° C. or less, in the absence of moisture or other chemically reactive conditions, for at least a week.

The terms “optional” or “optionally” as used throughout the specification, mean that the subsequently described event or circumstance can, but need not, occur, and that the description includes instances where the event or circumstance occurs and instances in which it does not. For example, “optionally substituted ring” means that the ring can, but need not be substituted, and the description includes situations where the ring is mono-, di-, tri-substituted with a substituent and situations where the ring is not substituted with a substituent.

A combination of substituents or variables is permissible only if such a combination results in a stable or chemically feasible compound. A stable compound or chemically feasible compound is one in which the chemical structure is not substantially altered when kept at a temperature of 4° C. or less, in the absence of moisture or other chemically reactive conditions, for at least a week.

As used herein, a “prodrug” means a compound that, after administration, is metabolized or otherwise converted to an active or more active form with respect to at least one property. To produce a prodrug, a pharmaceutically active compound (or a suitable precursor thereof) is modified chemically such that the modified form is less active or inactive, at least with respect to one biological property, relative to the pharmaceutically active compound, but the chemical modification is effectively reversible under certain biological conditions such that a pharmaceutically active form of the compound is generated by metabolic or other biological processes. A prodrug may have, relative to the drug, altered metabolic stability or transport characteristics, fewer side effects or lower toxicity, or improved flavor, for example (see the reference Nogrady, 1985, Medicinal Chemistry A Biochemical Approach, Oxford University Press, New York, pages 388-392). Prodrugs can also be prepared using compounds that are not drugs.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of cancer or a hyperproliferative disease, diminishment of extent of disease, delay or slowing of disease progression, amelioration, palliation or stabilization of the disease state, and other beneficial results described below.

As used herein, “reduction” of a symptom or symptoms (and grammatical equivalents of this phrase) means decreasing of the severity or frequency of the symptom(s), or elimination of the symptom(s).

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is meant to administering the drug to the patient.

As used herein, a “therapeutically effective amount” of a drug means an amount of a drug that, when administered to a subject with cancer or another hyperproliferative disease, will have the intended therapeutic effect, e.g., alleviation, amelioration, palliation or elimination of one or more manifestations of cancer or another hyperproliferative disease in the subject. The full therapeutic effect does not necessarily occur by administration of one dose, and may occur only after administration of a series of doses. Thus, a therapeutically effective amount may be administered in one or more administrations.

As used herein, a “prophylactically effective amount” of a drug means an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and may occur only after administration of a series of doses. Thus, a prophylactically effective amount may be administered in one or more administrations.

### 2. COMPOUNDS

The present invention provides compounds and methods for treatment of cancer. In order to appreciate the utility of such compounds and methods a brief description of cancer therapy can be useful.

Radiation therapy and surgical procedure used as first line of therapy against cancer is effective for cancer treatment at early and middle stages of localized cancer. The spread of cancer from its initial site to other parts of the body is called metastasis. Metastasis, which may not be effectively treated by surgery and/or radiatiob therapy, makes cancer fatal. Although there have been improvements in diagnosis, general patient care, surgical techniques, and local and systemic adjuvant therapies, cancer related mortality is still common.

Chemotherapy can be effective at all stages of the disease, but resistance to chemotherapy can lead to treatment failure. Drugs that suffer from resistance mechanism in cancer cells include the anthracyclines such as doxorubicin and daunorubicin. Doxorubicin and daunorubicin resistance occurs, among other factors, via multi-drug resistance where over-expressed p-glycoprotein removes the drug out of the cancer cells.

The compounds of the present invention are anthracycline analogs. In one aspect the present invention provides a compound having a structure of Formula (I): 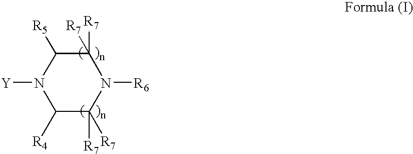
wherein Y is:

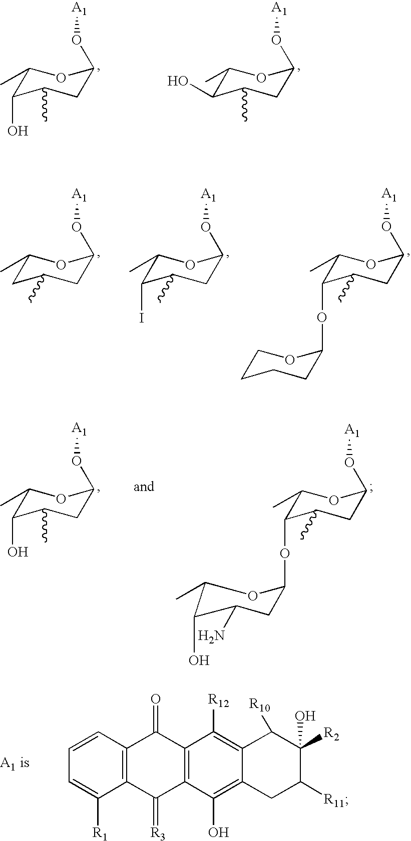

R₁ is hydrogen, C₁-C₆ alkyl or heteroalkyl, hydroxyl, C₁-C₆ alkoxy, amino, C₁-C₆ alkylamino, C₁-C₆ dialkylamino, mercapto, or C₁-C₆ alkylthio;

R₂ is selected from the group consisting of —CH₂CH₃, —COCH₃, —CH(OH)CH₃, —COCH₂OH, —CH(OH)CH₂OH, —C(═N-Z₁)-CH₃, and —C(═N-Z₁)-CH₂OH wherein Z₁ is —OZ₂ or —N(Z₂)₂ wherein each Z₂ is hydrogen, C₁-C₆ alkyl or heteroalkyl, C₃-C₈ cycloalkyl or heterocyclyl, and aryl or heteroaryl;

R₃ is O or NH;

R₁₀ and R₁₁ each independently is hydrogen, hydroxyl, or halogen;

R₁₂ is hydrogen or hydroxyl;

each n independently is 1-3;

R₄ and R₅ each independently is hydrogen, hydroxyl, C₁-C₆ alkoxy, cyano, amino, C₁-C₆ alkylamino, C₁-C₆ dialkylamino, mercapto, or C₁-C₆ alkylthio;

R₆ is —(CO₂)ₘ-Z₃ or —(CO)-Z₃ wherein m is 0 or 1 and Z₃ is hydrogen, C₁-C₆ alkyl or heteroalkyl, C₃-C₈ cycloalkyl or heterocyclyl, aryl or heteroaryl, C₁-C₆ alkylamino or di C₁-C₆ alkylamino and —C(Z₄)₂(CZ₄═CZ₄)₂Z₃ wherein each Z₄ is independently hydrogen,halogen, substituted or unsubstituted C₁-C₆ alkyl or heteroalkyl, C₁-C₆ aryl or heteroaryl, C₁-C₆ acyl or C₁-C₆ heteroacyl, aroyl, and heteroaroyl with the proviso that when n is 0 then Z₃ is not hydrogen;

each R₇ independently is hydrogen, substituted or unsubstituted C₁-C₆ alkyl or heteroalkyl, substituted or unsubstituted C₁-C₆ aryl or heteroaryl; and

an individual isomer or a racemic or non-racemic mixture of isomers, a pharmaceutically acceptable salt, solvate, hydrate, or a prodrug thereof.

In one embodiment, the present invention excludes the compounds

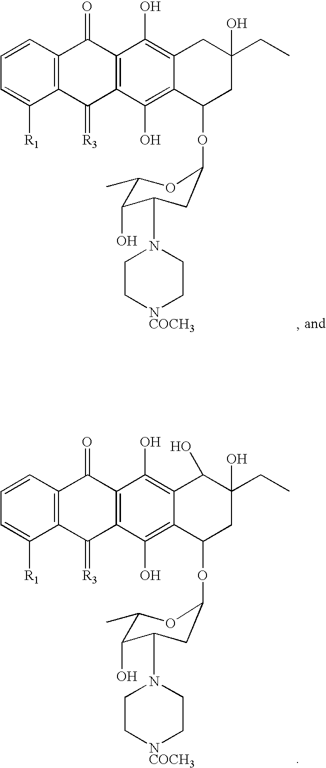

In another aspect, the present invention provides a compound having a structure of Formula (II):

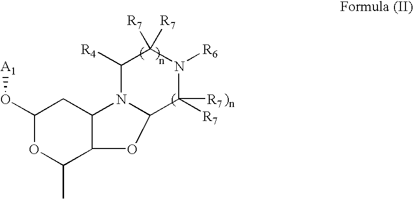

wherein A₁, n, R₁, R₂, R₃, R₄, R₆, and R₇ is defined as above; and

an individual isomer or a racemic or non-racemic mixture of isomers, a pharmaceutically acceptable salt, solvate, hydrate, or a prodrug thereof.

In one aspect the present invention provides a compound having a structure of Formula (III) 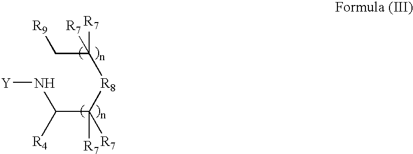
wherein Y and R₄-R₇ is defined as above;

R₈ is O, S, NR₆, or C(R₆)₂ wherein R₆ is defined as above; and

R₉ is halo, alkylsufonyloxy, heteroalkylsufonyloxy, arylsulfonyloxy, or heteroalkylsulfonyloxy; and

an individual isomer or a racemic or non-racemic mixture of isomers, a pharmaceutically acceptable salt, solvate, hydrate, or a prodrug thereof.

In another embodiment, the present invention provides compounds wherein Y is:

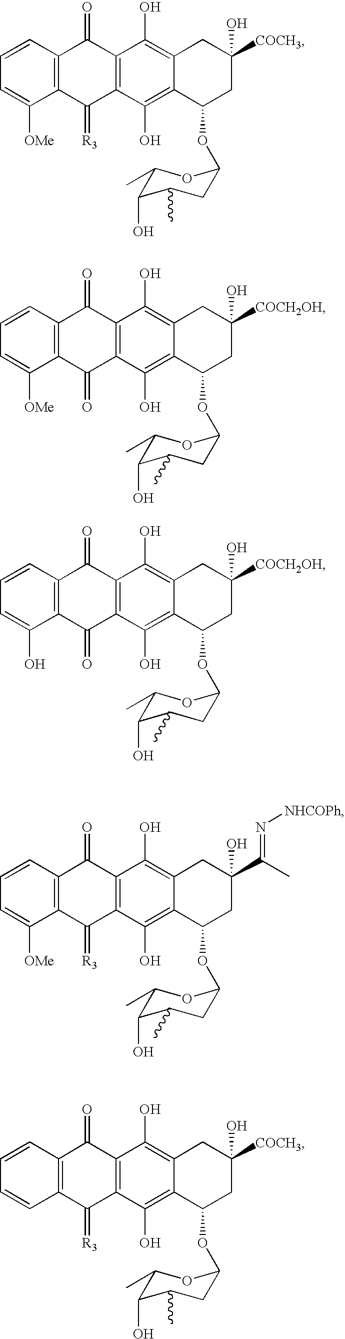

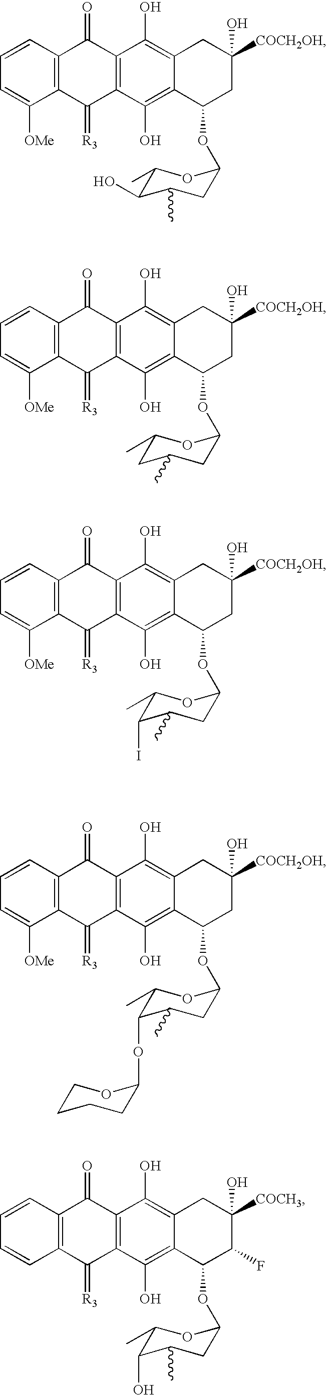

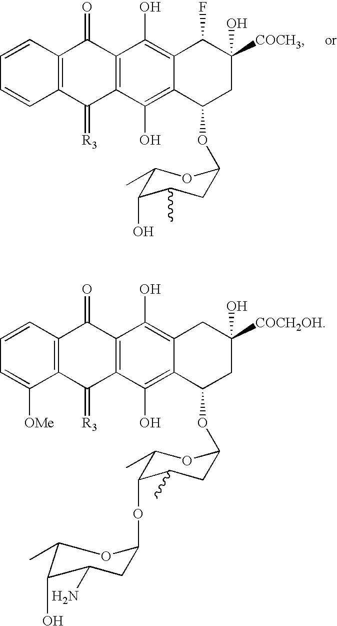

In another embodiment, the present invention provides compounds having structures of Formula (I)-(III) wherein A₁ is:

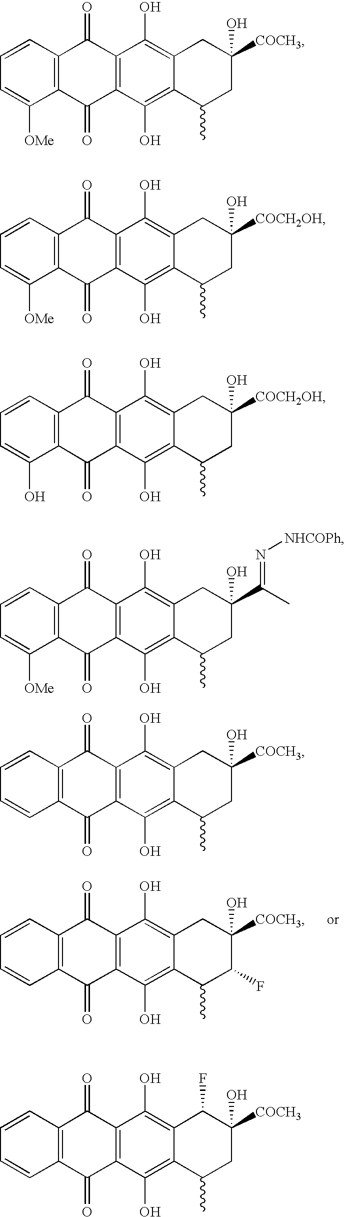

In one embodiment, the present invention provides compounds wherein each n is 1.

In another embodiment, the present invention provides compounds wherein R₂ is COCH₃, COCH₂OH, CH(OH)OH, or CH(OH)CH₂OH. In another embodiment, the present invention provides compounds wherein R₂ is COCH₃ or COCH₂OH. In another embodiment, the present invention provides compounds wherein each R₄ and R₅ independently is hydrogen, hydroxyl, methoxy, or cyano.

In another embodiment, the present invention provides compounds wherein R₁ is OMe or H, R₃ is O, R₁₂ is hydroxyl, each of R₁₀ and R₁₁ is hydrogen.

In another embodiment, R₆ is COCF₃, COCH₃, COCH₂CMe₃, CO₂CMe₃, CO₂CH₂CH₂OMe, CO₂CH₂CMe₃,

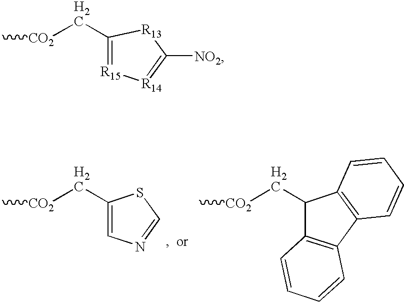

wherein R₁₃ is O, S, or NR₁₆;

each R₁₄ and R₁₅ independently is N or CH; and

R₁₆ is (C₁-C₆) alkyl or heteroalkyl.

In another embodiment, R9 is OTs and Cl.

In another embodiment, the present invention provides compounds having the structures of Formula (IV)-(VI): 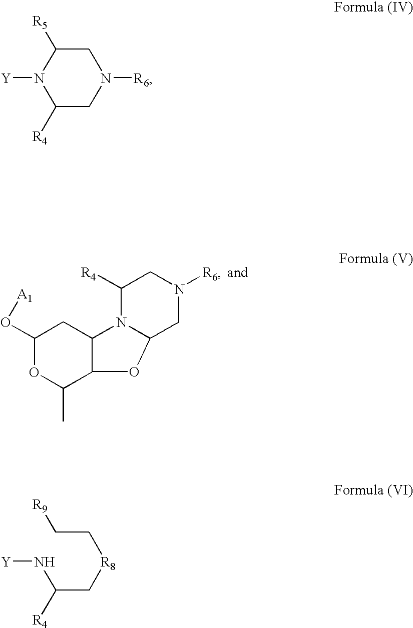
wherein Y, R₄-R₆, R₈, and R₉ are defined as above. In another embodiment, the present invention provides compounds having the structures of Formula (IV) and (VI) wherein Y is 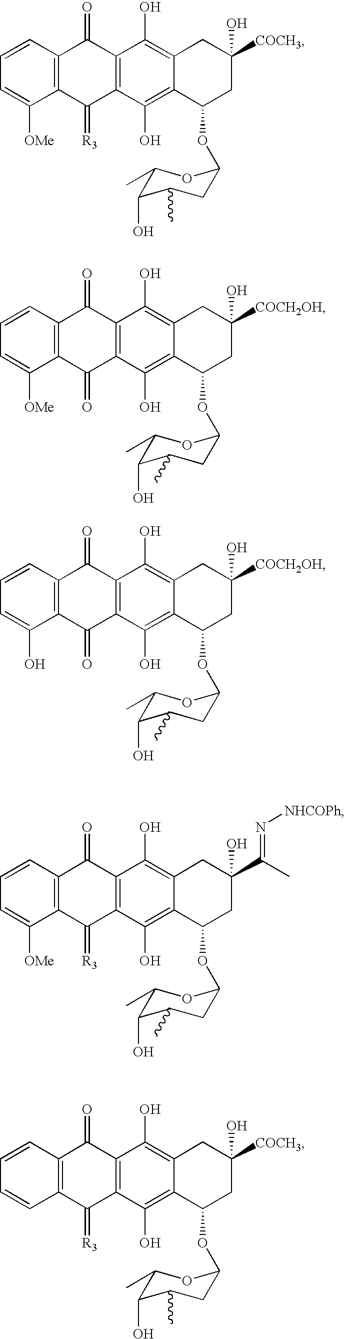 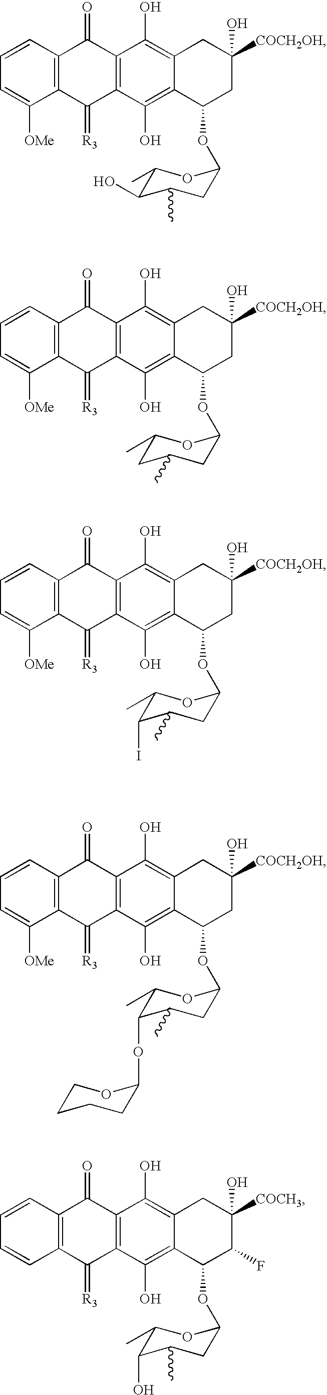 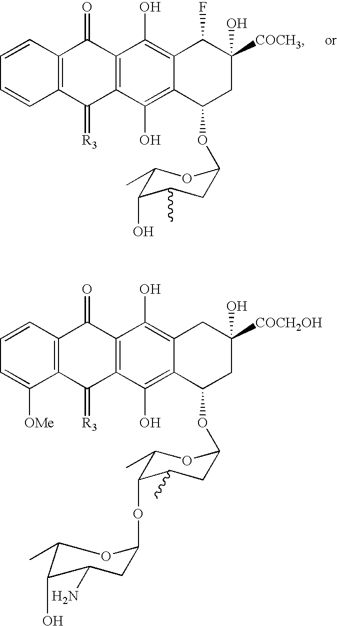
and A₁ is:

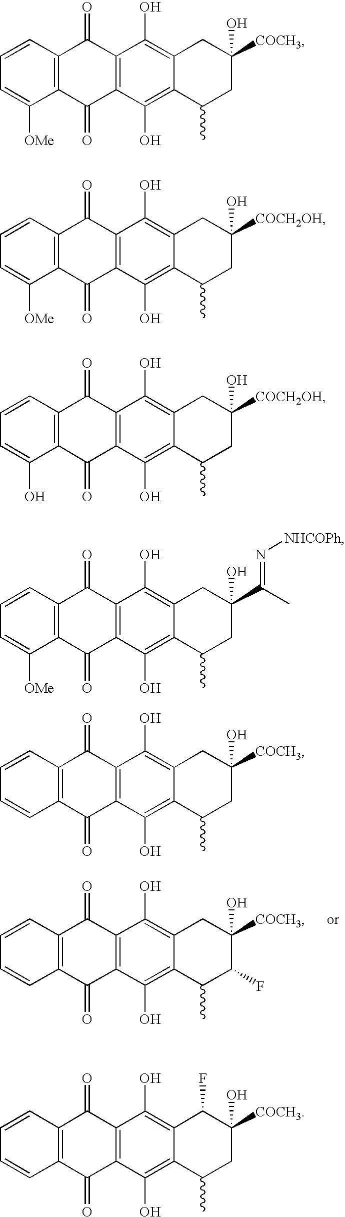

In another related embodiment, Y is:

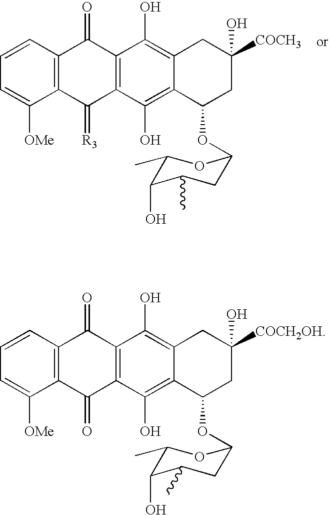

In another related embodiment, the present invention provides the compound having structure of formula (IV) wherein Y is:

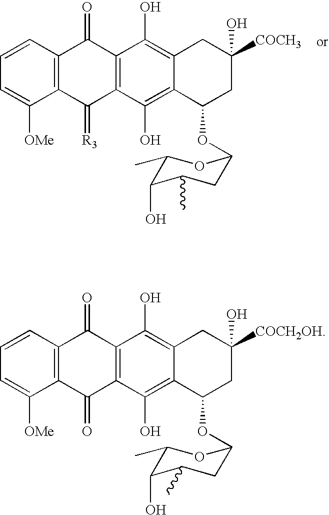

In another embodiment, the present invention provides the compound having structure of formula (V) wherein A₁ is:

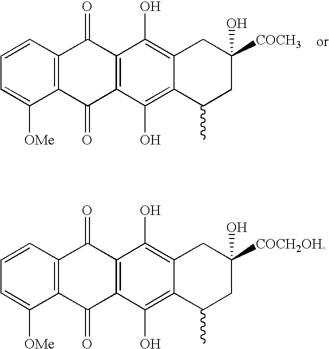

In one embodiment, the present invention provides the following compounds: 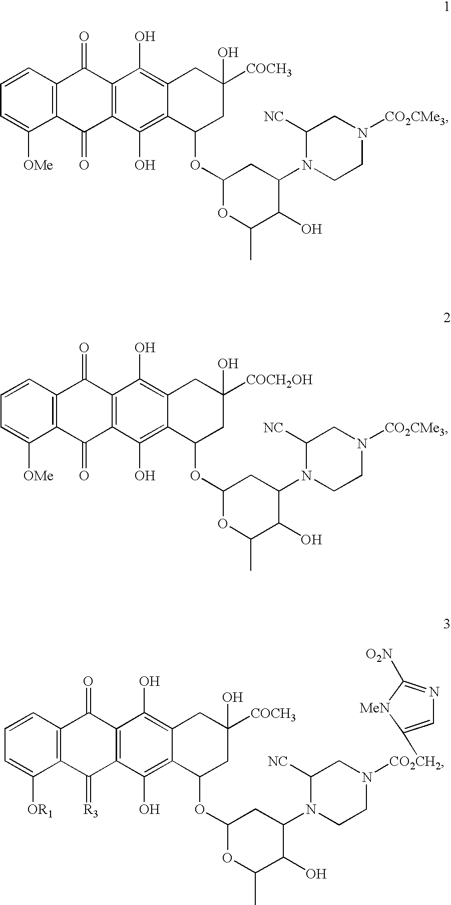 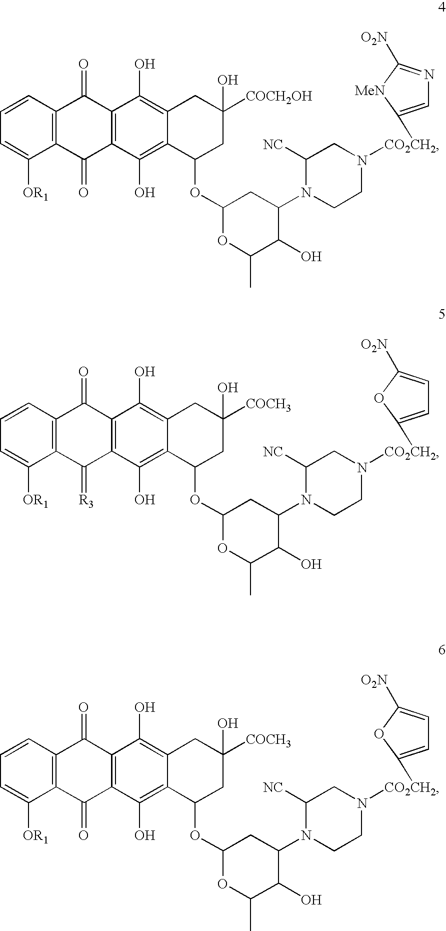 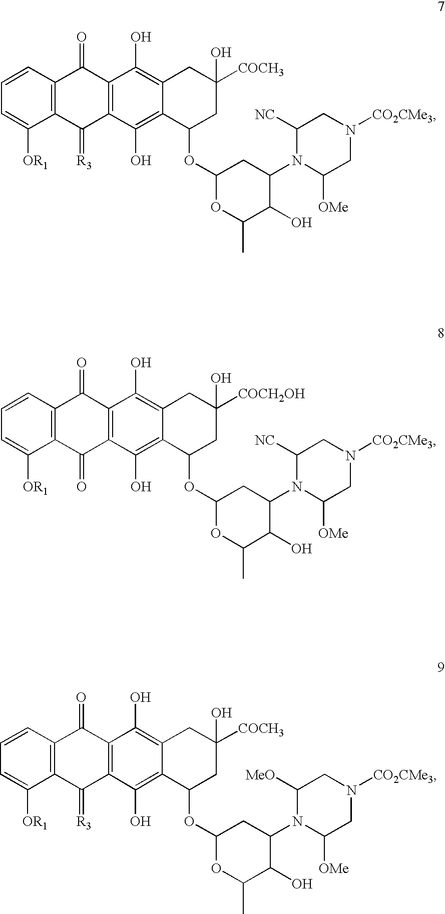 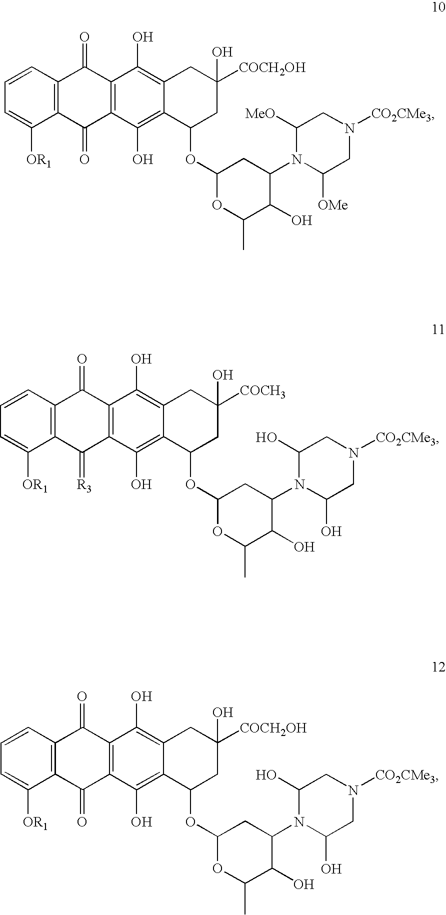 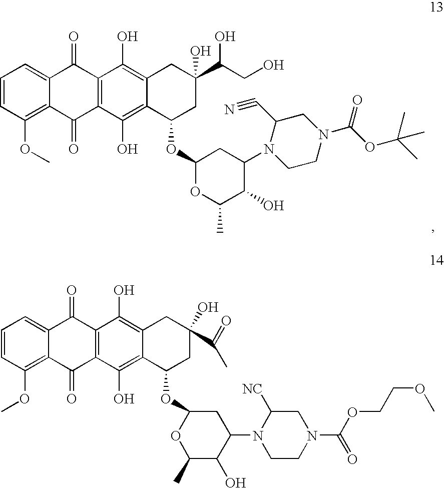 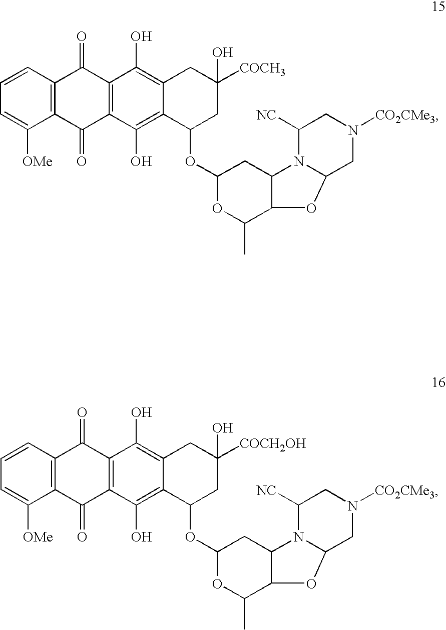 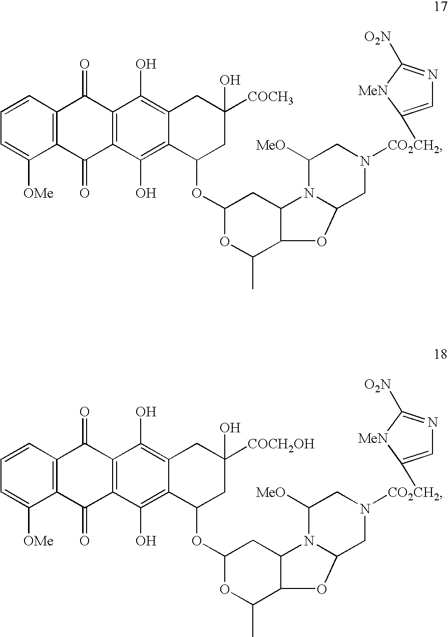 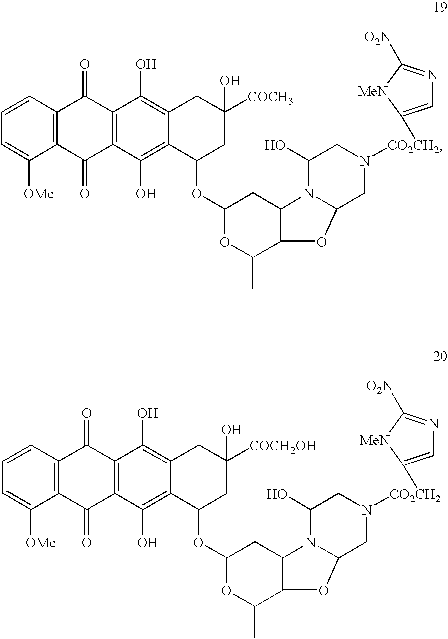 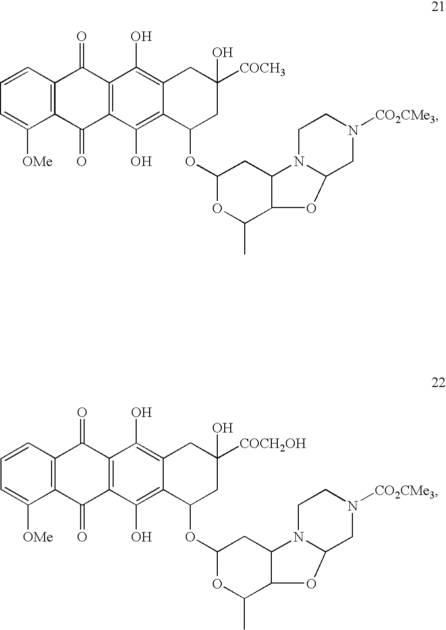 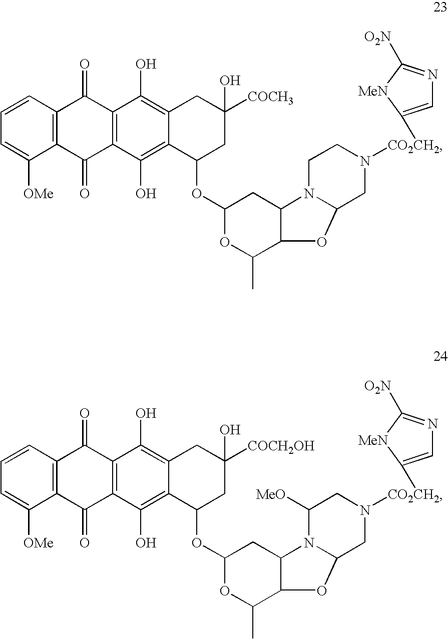 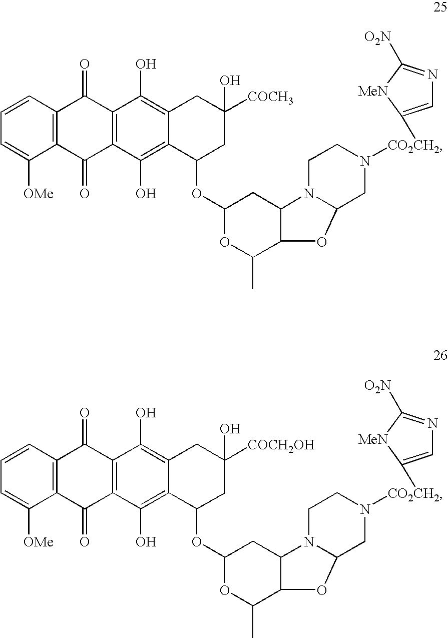 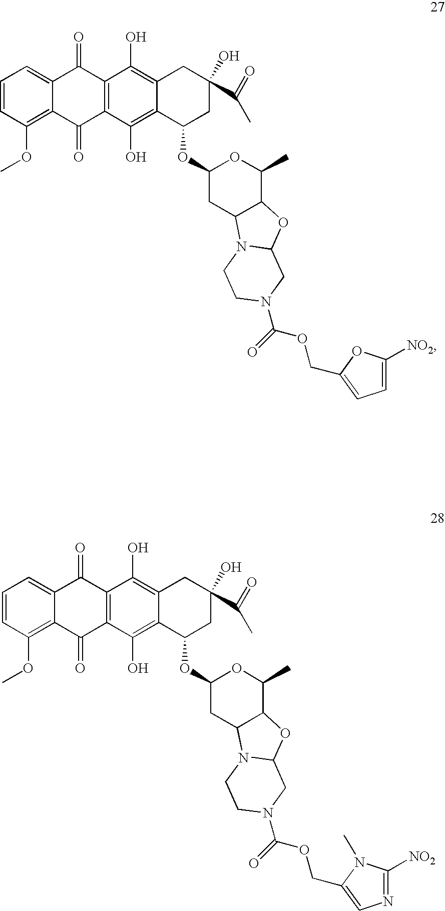 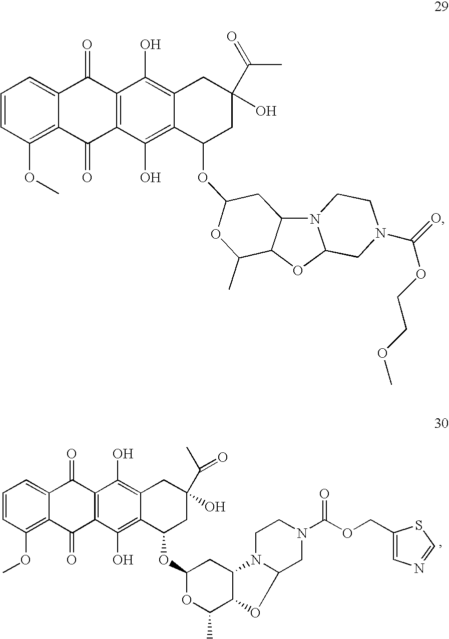 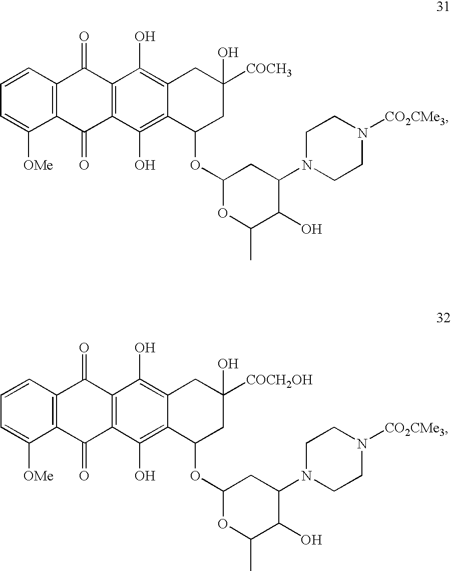 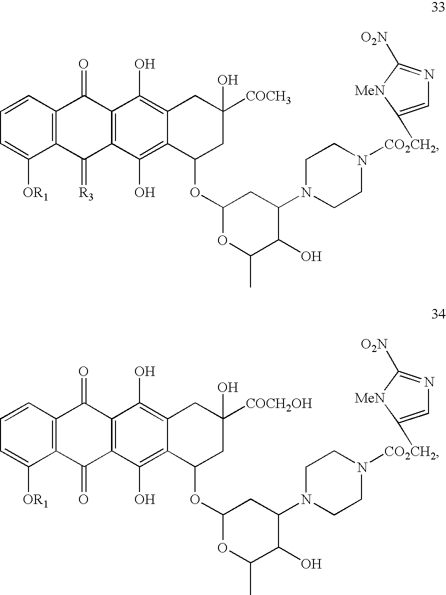 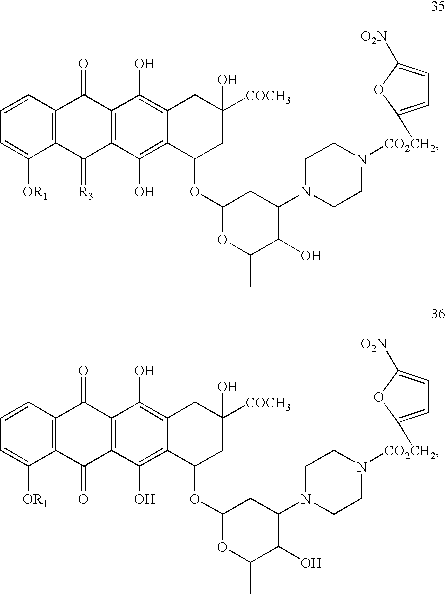 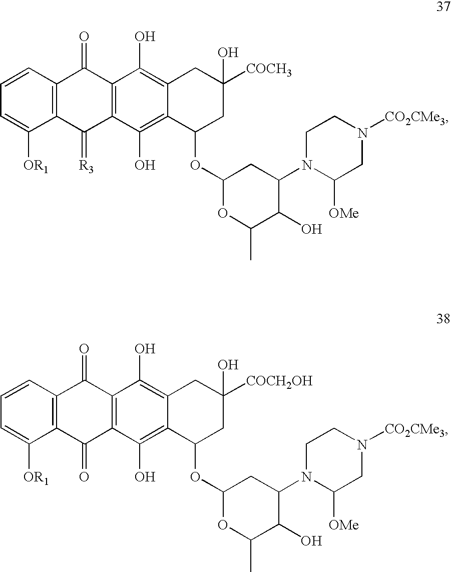 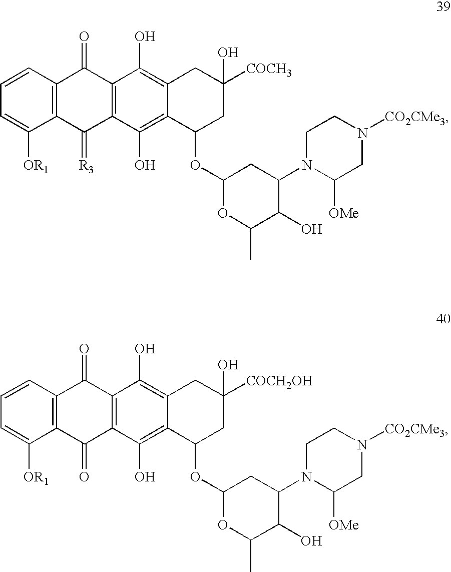 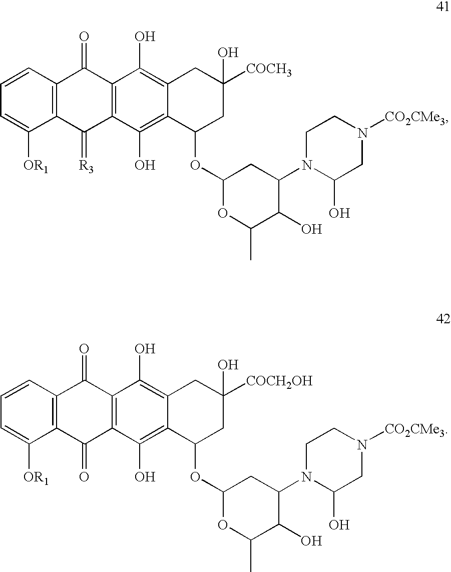 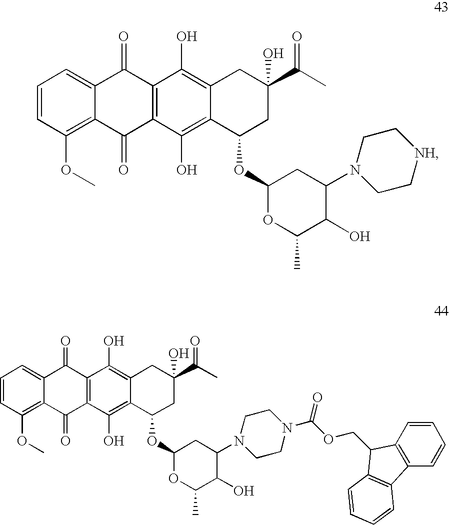 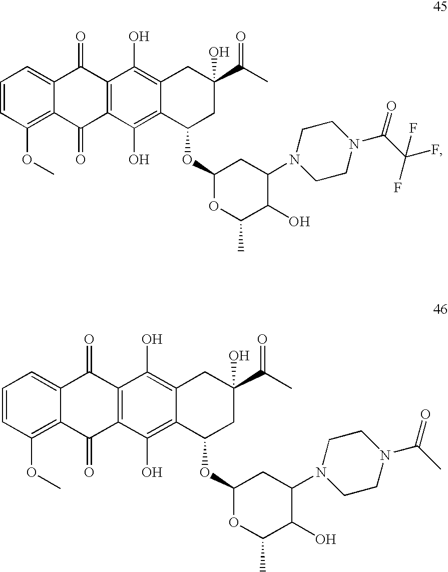 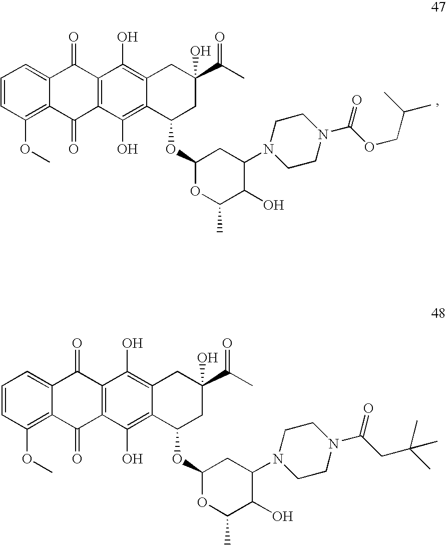 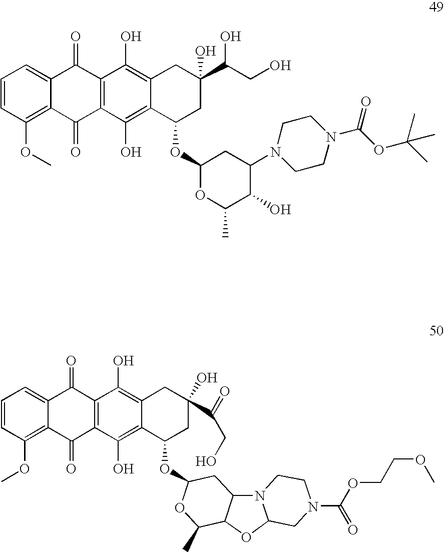 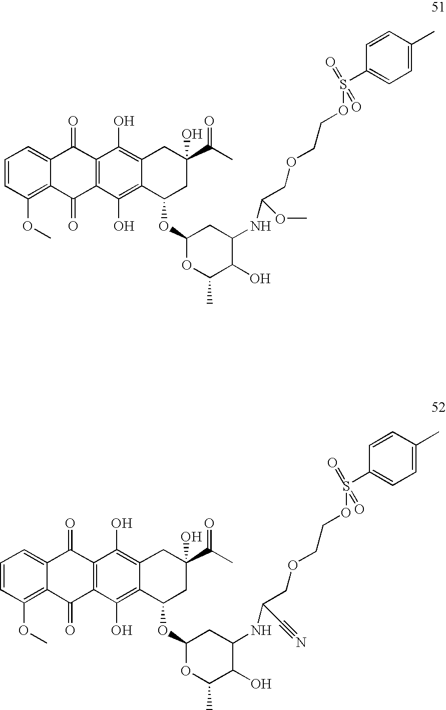 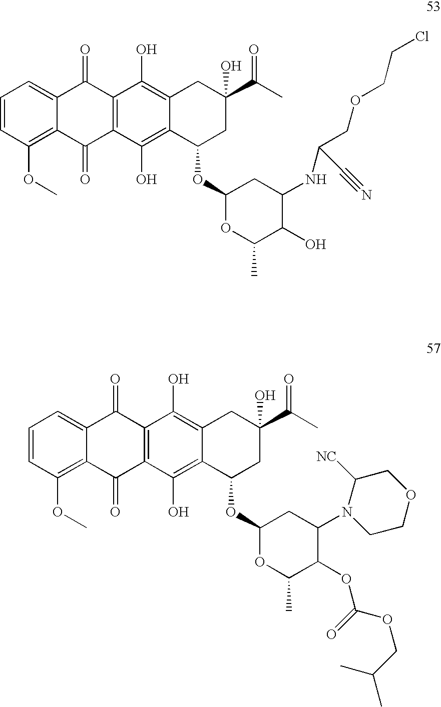 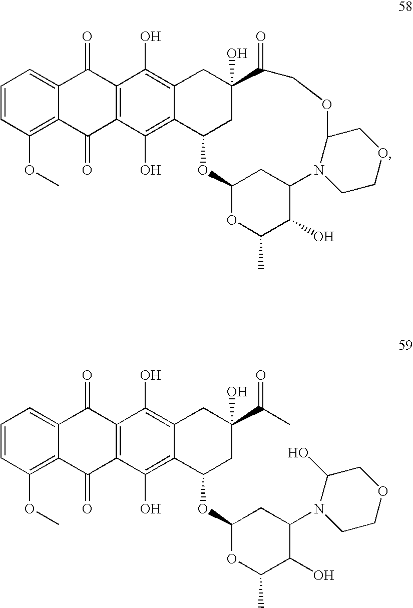 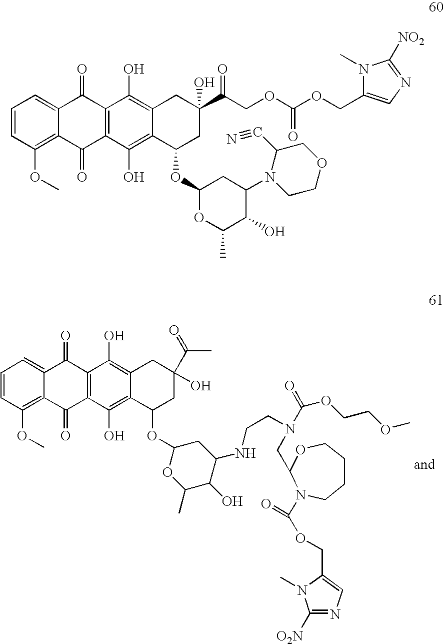
Compounds 64-71 described in the EXAMPLES section.

In another aspect, the present invention provides an anthracycline analog-protein bioconjugate wherein the anthracycline analog is selected from compounds having structures of Formulas (I)-(VI). In another embodiment, the protein is an antibody. In another embodiment, the present invention provides a tumor specific anthracycline analog-antibody bioconjugate. In another embodiment, the anthracycline analog has a cytotoxicity of about 0.1 picoM-1 μM and about 1 picoM to 100 nM. In another embodiment, the anthracycline analog-protein bioconjugate having a cytotoxicity of about 0.1 picoM-1 μM is synthesized by employing an anthracycline having cytotoxicity about 5-1000, about 10-100, and about 50 folds less than the anthracycline analog-protein bioconjugate, and a suitably modified protein synthesized from the protein employing synthetic methods described in the following section and using known methods of bioconjugation.

### 3. METHODS OF SYNTHESIS

In another aspect the present invention provides a method of making the compounds having structures of Formulas (I)-(III): 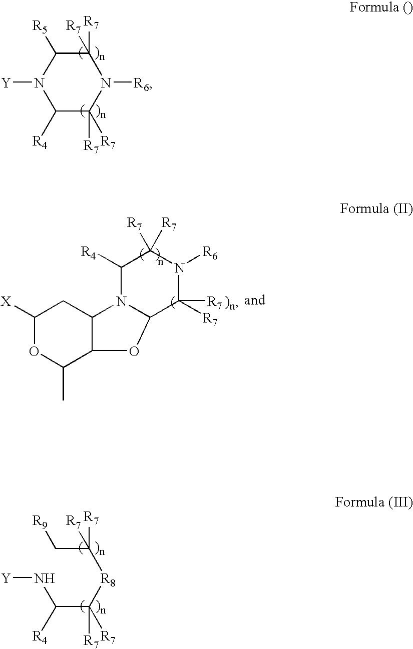
the method comprising reacting Y—NH₂ with 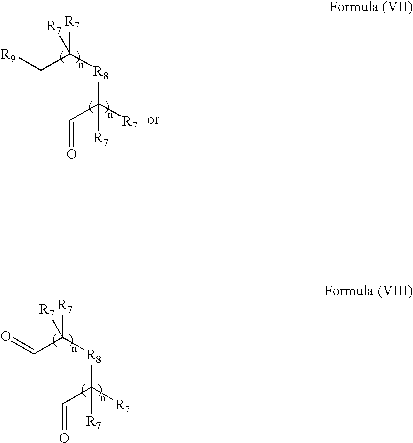
and optionally a cyanide salt wherein Y, n, R₆, R₇, and R₉ are defined as in any one of Formulas (I)-(VI). In one embodiment, the Compound having structure of Formula (VII) is:

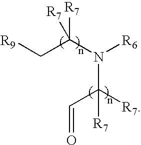

In another embodiment, the Compound having structure of Formula (VIII) is

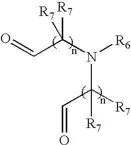

In another embodiment, the method further comprises reacting a reducing agent. In another embodiment, the reducing agent is a borohydride or a borohydride derivative. In another embodiment, the borohydride derivative employed is triacetoxyborohydride or cyanoborohydride.

### 4. METHODS OF TREATMENT

A. Treatment of Cancer

In one aspect the present invention provides a method for treating cancer or other hyperproliferative disease comprising administering a therapeutically effective amount of a compound of the present invention to a patient or subject in need of such therapy. In one embodiment, the disease treated in cancer. Generally, the subject can be any human or non-human mammal. Particularly, a subject is a human subject. Other subjects include but are not limited to non-human primates, dogs, cats, farm animals and horses. In one embodiment, the compound of the present invention is administered alone. In another embodiment, the compound of the present invention is administered in combination with one or more additional anti-cancer agents. In another embodiment, the compound of the present invention is administered in conjunction or combination with a therapeutic cancer treatment: including but not limited to surgery and radiation. The compound of the present invention will typically be administered in a pharmaceutical composition. Various pharmaceutical compositions that can be used are described in the Formulation section infra.

The compounds of the present invention and their pharmaceutical compositions can be used to treat any type of cancer in a patient or a subject, particularly in a human patient or subject. Cancers that can be treated include, but are not limited to, leukemia, breast cancer, skin cancer, bone cancer, liver cancer, brain cancer, cancer of the larynx, gallbladder, pancreas, rectum, parathyroid, thyroid, adrenal, neural tissue, head and neck, stomach, bronchi, kidneys, basal cell carcinoma, squamous cell carcinoma of both ulcerating and papillary type, metastatic skin carcinoma, osteosarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic corneal nerve tumor, marfanoid habitus tumor, Wilm's tumor, seminoma, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas.

B. Treatment of Hyperproliferative Diseases

In one aspect the present invention provides a method for treating a noncancer hyperproliferative disease comprising administering a therapeutically effective amount of a compound of the present invention to a patient in need of such therapy. In one embodiment, the hyperproliferative disease is selected from the group consisting of angiofibroma, atherosclerosis, benign prostatic hyperplasia, corneal graft rejection, gout, graft versus host disease, glaucoma, inflammatory diseases such as inflammatory bowel disease, ischemic heart and peripheral vascular disease, Karposi's sarcoma, keloids, life threatening infantile hemangiomas, macular degeration, myocardial angiogenesis, myocardial infraction, multiple sclerosis, neovascular-based dermatological conditions, Osler-Webber Syndrome, osteoarthritis, psoriasis, psoriatic arthritis, pulmonary fibrosis, psoriasis, rheumatoid arthritis, restenosis, rheumatoid arthritis, scleroderma, telangectasia, and wound granularization.

C. Administration, Dosage, and Formulation

The compounds of the present invention will typically be formulated as pharmaceutical formulations for administration to a subject. Described in this section are modes of administration, formulations, and dosages that may be used when treating cancers using the compounds provided herein.

Administration of the compounds of the present invention for the treatment of cancer can be effected by any method that enables delivery of the compounds of the invention to the cancer cells. Many cancer drugs are administered by intravenous injection, and the compounds of the present invention can be formulated for such administration, including not only ready-for-injection formulations but also lyophilized or concentrated formulations that must be rehydrated or diluted, respectively, prior to injection. In addition to these formulations, the compounds of the present invention can be formulated for administration by oral routes, intraduodenal routes, parenteral injection (including intravenous, subcutaneous, intramuscular, intravascular or infusion), topical, and rectal routes. The actual route of administration and corresponding formulation of the compounds of the present invention will depend, among other factors, on the type of cancer being treated, the compound of the present invention selected for administration, the severity of the cancer, and the age, weight, and condition of the patient,.

The amount of the compound of the present invention administered, and thus the amount of the compound of the present invention contained in the dose administered and the product comprising that dose, will be dependent on the subject being treated, the severity of the cancer, localization of the cancer, the rate of administration, the of the compound of the present invention used in treatment, and the discretion of the prescribing physician. However, a therapeutically effective dosage is typically in the range of about 0.001 to about 100 mg per kg body weight, or in one embodiment of the present invention, about 1 to about 35 mg/kg/day, in single or divided doses. For a 70 kg human, this would amount to about 0.05 to about 7 g/day, or in one embodiment of the present invention, about 0.2 to about 2.5 g/day. In some instances, dosage levels below the lower limit of the aforesaid range may be more than adequate, while in other cases still larger doses may be employed without causing any harmful side effect; larger doses can also be divided into several small doses for administration throughout the day.

In another aspect, the present method provides a pharmaceutical composition or formulation comprising a compound of the present invention and a pharmaceutically acceptable carrier or diluent. A formulation of a compound of the present invention can, for example, be in a form suitable for oral administration as a tablet, capsule, pill powder, sustained release formulation, solution, and suspension; for parenteral injection as a sterile solution, suspension or emulsion; for topical administration as an ointment or cream; and for rectal administration as a suppository. A formulation of a compound of the present invention can be in unit dosage forms suitable for single administration of precise dosages and will typically include a conventional pharmaceutical carrier or excipient.

Suitable pharmaceutically acceptable carriers include inert diluents or fillers, water and various organic solvents. The pharmaceutical compositions can, if necessary, contain additional ingredients such as flavorings, binders, excipients, and the like. Thus for oral administration, tablets containing various excipients, such as citric acid may be employed together with various disintegrants, such as starch, alginic acid, and certain complex silicates, and with binding agents such as sucrose, gelatin and acacia. Additionally, lubricating agents such as magnesium stearate, sodium lauryl sulfate, and talc can be used to prepare the tablet forms of formulations of the compounds provided herein. Solid compositions of a similar type can be employed in soft and hard filled gelatin capsules. Particular materials, therefore, include lactose or milk sugar and high molecular weight polyethylene glycols. When aqueous suspensions or elixirs are desired for oral administration, the compound of the invention therein may be combined with various sweetening or flavoring agents, coloring matters or dyes and, if desired, emulsifying agents or suspending agents, together with diluents such as water, ethanol, propylene glycol, glycerin, or combinations thereof.

Exemplary parenteral administration forms include solutions or suspensions of the compound of the present invention in sterile aqueous solutions, for example, aqueous polyethylene glycols, aqueous propylene glycol, saline, or dextrose solutions. Such dosage forms can be suitably buffered, if desired.

Methods of preparing various pharmaceutical compositions with a specific amount of active drug are known, or will be apparent, to those skilled in this art in view of this disclosure. For examples, see Remington's Pharmaceutical Sciences, Mack Publishing Company, Philadelphia, Pa., 17^{th} Edition (1984).

D. Treatment Combinations

In one aspect, the present invention provides a method of treating cancer by administering a compound of the present invention in combination with an effective amount of one or more chemotherapeutic agents, an effective amount of radiotherapy, an appropriate surgery procedure, or any combination of such additional therapies.

When a compound of the present invention is administered in combination with one or more of the additional therapies, the compound of the present invention and additional therapy can be administered at the same time or can be administered separately. For example, if a compound of the present invention is administered with an additional chemotherapeutic agent (hereinafter “agent”), the two agents can be administered simultaneously or can be administered sequentially with a time difference between administrations. One of skill in the art upon reading this specification will appreciate various methods of administering the compound of the present invention and the agent or agents simultaneously and sequentially and possible time difference between administrations. The agents can be administered as the same or different formulations and may be administered via the same or different routes.

Chemotherapeutic agents that can be used in combination with the compound of the present invention include but are not limited to busulfan, improsulfan, piposulfan, benzodepa, carboquone, 2-deoxy-D-glucose, lonidamine and analogs thereof, glufosfamide, meturedepa, uredepa, altretamine, imatinib, triethylenemelamine, triethylenephosphoramide, triethylenethiophosphoramide, trimethylolomelamine, chlorambucil, chlornaphazine, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-fluorouracil, tegafur, L-asparaginase, pulmozyme, aceglatone, aldophosphamide glycoside, aminolevulinic acid, amsacrine, bestrabucil, bisantrene, carboplatin, defofamide, demecolcine, diaziquone, elfornithine, elliptinium acetate, etoglucid, flutamide, gallium nitrate, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, teniposide, tenuazonic acid, triaziquone, 2,2′,2″-trichlorotriethylamine, urethan, vinblastine, cyclophosphamide, and vincristine. Other chemotherapeutic agents that can be used include platinum derivatives, including but not limited to cis platinum, carboplatin, oxaliplatin, and oxoplatin.

### 5. EXAMPLES

A. Synthesis of Anthracycline Analogs

General Procedure (I): Preparation of Compound A Employing NaBH₃CN as a Reducing Agent

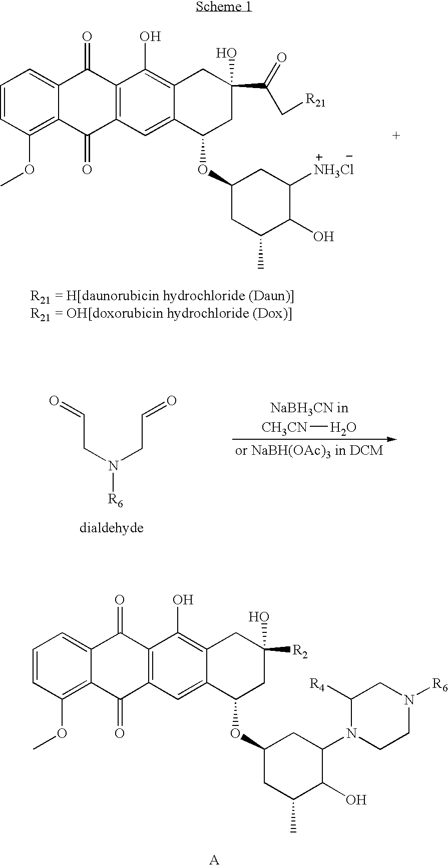

A solution of the dialdehyde (20-30 eq.) in 5 mL of acetonitrile-H₂O (1:1) was adjusted to pH 7.0 with a solution of NaHCO₃. The above solution was treated with a solution of NaBH₃CN (1-5 eq.) in 0.5 mL of acetonitrile-H₂O (1:1) and then with a solution of Daun (R₁═H) or Dox (R₁═OH) (1 eq.) in 1 mL of acetonitrile-H₂O (1:1) rt. After 1-2 hrs, the reaction mixture was worked up by dilution with 10 mL water and extraction with DCM 10 mL×3. The organic phase was washed with water, dried over Na₂SO₄, and concentrated under reduced pressure. Chromatography afforded compound A.

Synthesis of Compound 31 (R₆=Boc R₂═COCH₃, R₄═H) and Compound 31i (R₆=Boc, R₂═CH(OH)CH₃, R₄═CN)

The general procedure (I) was followed using crude dialdehyde (30 eq.), NaBH₃CN (1 eq.), and Daun (1 eq.). After work-up, chromatography (DCM:MeOH=100:15(V/V)) of the residue on silica gel afforded Compounds 31 and 31i.
Synthesis of Compound 3 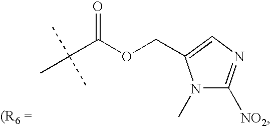
R₂═COCH₃, R₄═CN)

The general procedure was followed using general procedure (I) crude dialdehyde (25 eq.), NaBH₃CN (1 eq.), and Daun (1 eq.). After work-up, chromatography (DCM:MeOH=100:15(V/V)) of the residue on silica gel afforded Compound 3.

Synthesis of Compound 32 (R₆=Boc, R₂═COCH₂OH, R₄═H), Compound 32i(R₆=Boc, R₂═CH(OH)CH₂OH, R₄═CN), and Compound 32ii (R₆═Boc, R₂═CH(OH)CH₂OH, R₄═H)

The general procedure (I) was followed using, crude dialdehyde (25 eq.), NaBH₃CN (5 eq.), and Dox (1 eq.). After work-up, the residue was dissolved in DCM and purified by preparative TLC (DCM:MeOH=10:1(v/v)) to yield Compounds 32-32ii.

General Procedure (II): Preparation of Compound A Employing NaBH(OAc)₃ as a Reducing Agent

A mixture of the dialdehyde (2-3 eq.) and daunorubucin (R₂₁═H) (1 eq.) in 5 mL of dry DCM was stirred at rt for 10 min. After the reaction mixture was cooled down to 0° C., NaBH(OAc)₃ (5-6 eq.) was added. The temperature of the reaction mixture was raised from 0° C. to rt in 1 h while stirring, water (10 mL) added to the reaction mixture and extracted with DCM (10 mL×3). The organic phase was washed with H₂O, 5% NaHCO₃, and brine, dried over Na₂SO₄, and concentrated under reduced pressure to yield a residue which was separated by flash chromatography on silica gel to yield compound A.

Synthesis of Compound 44 (R₆=Fmoc, R₂═COCH₃, R₄═H) and Compound 43 (R₆═R₄═H, R₂═COCH₃)

General procedure (II) was followed using, crude dialdehyde (3 eq.). Purification was achieved by flash column chromatography (DCM: MeOH=100:5(v/v)) to yield compound 44.

Compound 44 was added to a solution of (2 M) dimethylamine in THF and stirred at rt for 1 h. After removing solvent under reduced pressure, MeOH (5 mL) was added and removed again. The residue was washed with ether to yield Compound 43:
Synthesis of Compound 33 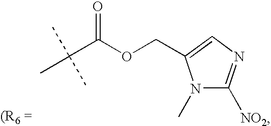
R₂═COCH₃, R₄═H):

General procedure (II) was followed using dialdehyde (2 eq.). Purification was achieved by flash column chromatography (DCM: MeOH=100:5(v/v)) to yield Compound 33.

Synthesis of 45 (R₆═COCF₃, R₂═COCH₃, R₄═H)

General procedure (II) was followed using crude dialdehyde (2.5 eq.). Purification was achieved by flash column chromatography (DCM: MeOH=100:5(v/v)) to yield Compound 45.

Synthesis of Compound 46 (R₆═COMe, R₂═COCH₃, R₄═H)

General procedure (II) was followed using crude dialdehyde (3 eq.). Purification was achieved by flash column chromatography (AcEt: MeOH=100:2(v/v)) to yield Compound 46.

Synthesis of Compound 47 (R₆═CO₂CH₂CHMe₂, R₂═COCH₃, R₄═H)

General procedure (II) was followed using crude dialdehyde (3 eq.), NaBH(OAc)₃ (5 eq.). Purification was achieved by flash column chromatography (Hex: AcOEt=10:80˜0:100(v/v)) to yield Compound 47.

Synthesis of Compound 48 (R₆═COCH₂CMe₃, R₂═COCH₃, R₄═H)

General procedure (II) was followed using crude dialdehyde (3.5 eq.), NaBH(OAc)₃ (5 eq.). Purification was achieved by flash column chromatography (Hex: AcOEt=10:80˜0:100(v/v)) to yield Compound 48.
General Procedure (III): Synthesis of Compound 29

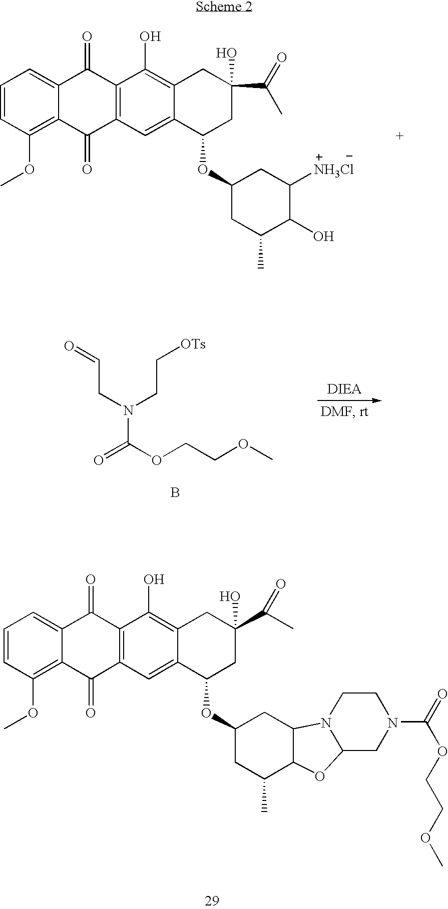

DIEA (2 eq.) was added to a solution of Daun (1 eq.) and B (1 eq.) in 3 mL dry DMF at rt. The mixture was stirred at rt overnight. The reaction mixture was diluted with 10 mL water and extracted with DCM (10 mL×2). The organic phase was washed with H₂O, 10% NaHCO₃, and brine, dried over Na₂SO₄, and concentrated under reduced pressure. Flash chromatography of the residue on silica gel (DCM: MeOH=100:5(v/v)) afforded Compound 29.
Synthesis of Compound 61

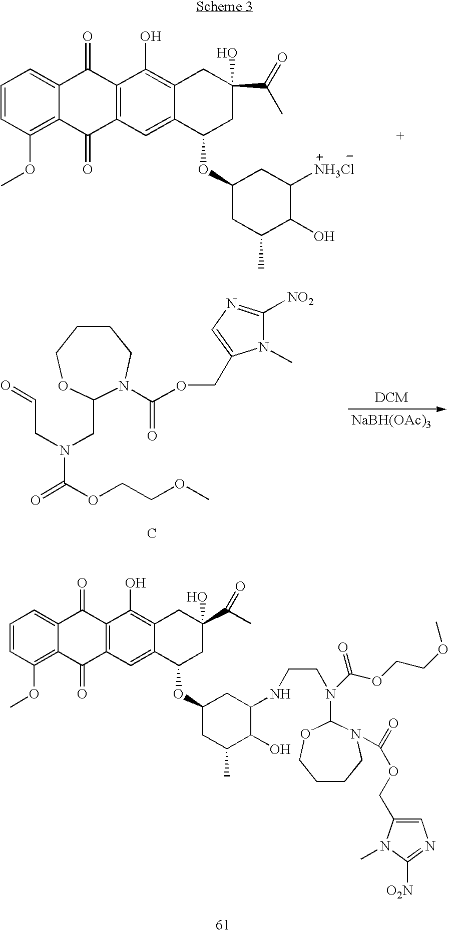

General procedure (II) was followed using compound C (1.5 eq.), Daun (1 eq.) NaBH(OAc)₃ (2 eq.). Purification was achieved by purified by preparative TLC (DCM: MeOH=10:1(v/v)) to give Compound 61.
Synthesis of Compound 53

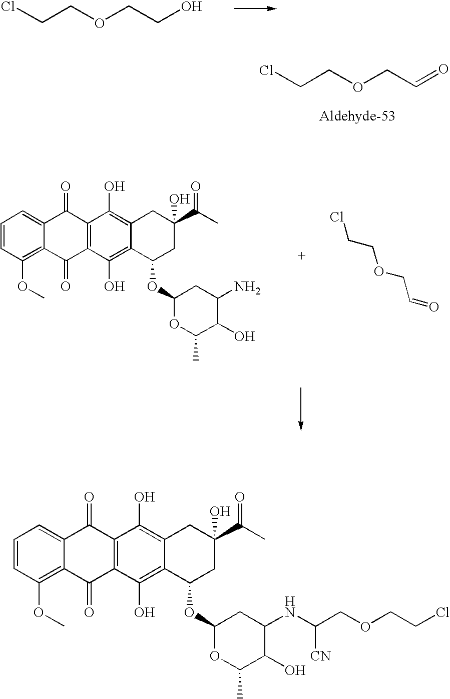

To a solution of 2-(2-Chloroethoxy)-ethanol (0.8 mL, 7.55 mmol) in 8 mL DCM, was added drop wise Des-Martin periodinane and the reaction mixture stirred at roomtemperature (rt) for 3 hours to yield Aldehyde-53. To a solution of daunorubicin (100 mg, 0.177 mmol) in 3 mL Methanol/Ethyl acetate (1:1), was added KCN (1 eq, 11.5 mg) and Aldehyde-53 (1 eq, 22 mg) and the reaction mixture stirred for 3 hours. Separation by flash column chromatography (eluent: MeOH/DCM 15:85) yielded compound 53.
Synthesis of Compound 64

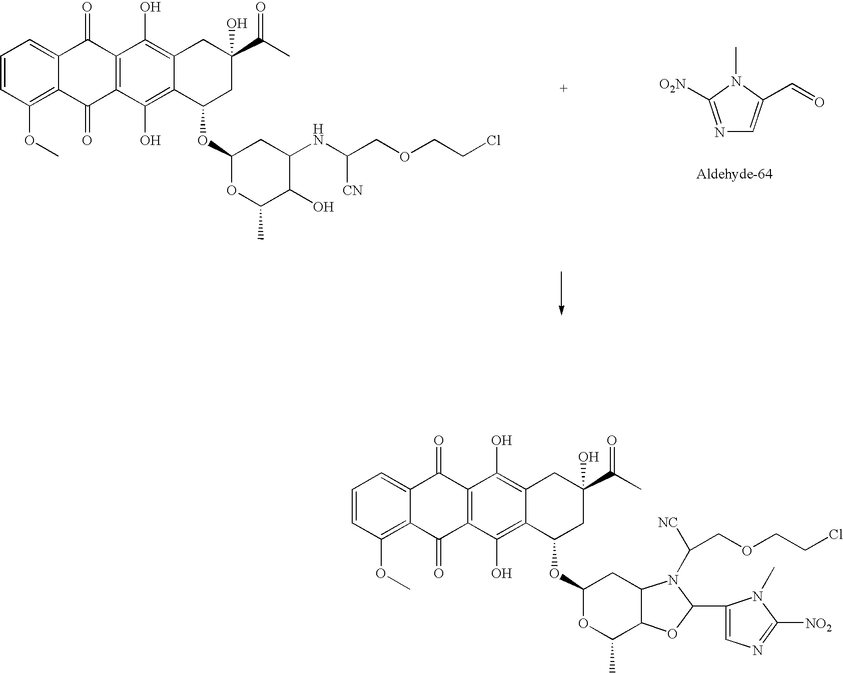

To Compound 53 (5 mg, 0.007 mmol) in dichloromethane (DCM, 0.5 mL) was added aldehyde-64 (1 eq, 1.16 mg), and TsOH (1 eq, 1.3 mg), and the reaction mixture was stirred at room temperature for 2 hours. Separation by flash column chromatography (eluent: MeOH/DCM 15:85) yielded compound 64.
Synthesis of Compound 65

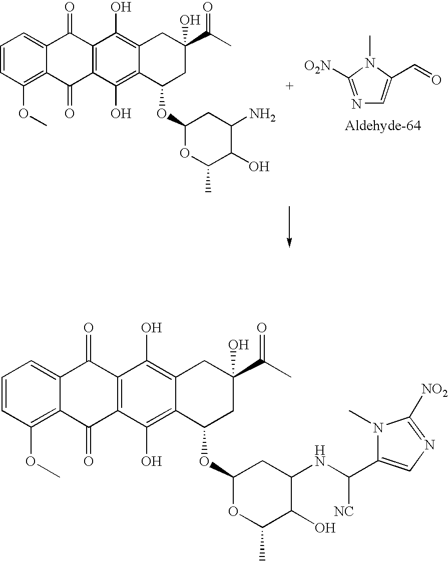

To daunorubicin (10 mg, 0.017 mmol) was added ethanol (0.5 mL), Aldehyde-64 (1.2 eq, 3.12 mg, 0.02 mmol), and KCN (1 eq, 1.15 mg, 0.017 mmol) and the reaction mixture was stirred at room temperature for 3 hours. Separation by flash column chromatography (MeOH/DCM 15:85) yielded compound 65.
Synthesis of Compound 59

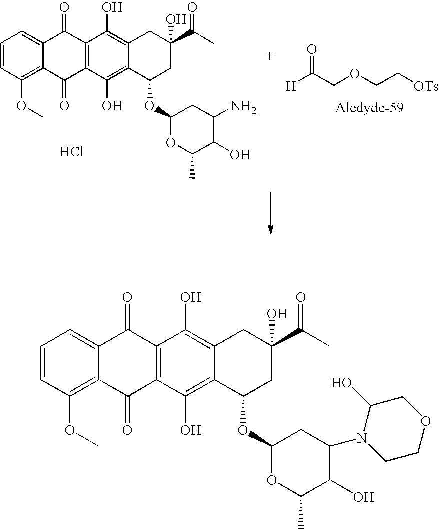

A solution of daunorubicin hydrochloride (50 mg, 0.088 mmol) in N,N-dimethylformamide (2 mL), Aledyde-59 (1 eq, 0.088 mmol, 22.8 mg), and N,N-Diisopropylethylamine (2 eq, 0.17 mmol, 0.03 mL) was stirred at room temperature overnight and then warmed up to 40° C. for 30 minutes. Separation by flash column chromatography (eluent: MeOH/DCM 10:90) yielded Compound 59.
Synthesis of Compound 58

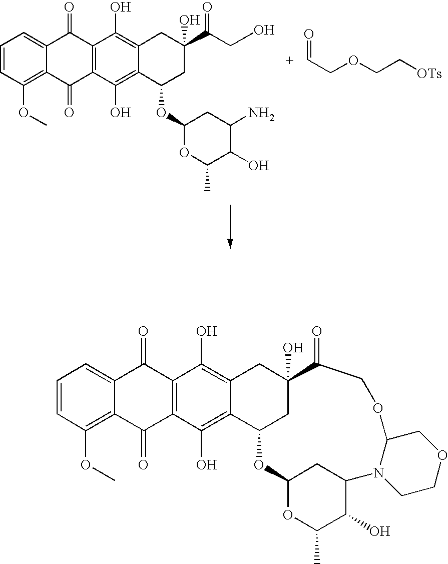

To a solution of doxorubicin (50 mg, 0.086 mmol) in DMF (2 mL) was added Aledyde-59 (0.5 eq, 11.1 mg) and DIEA (3 eq, 0.045 mL), and stirred at rt overnight. Separation by flash column chromatography (eluent: MeOH/DCM 10:90) yielded Compound 58.
Synthesis of Compound 66

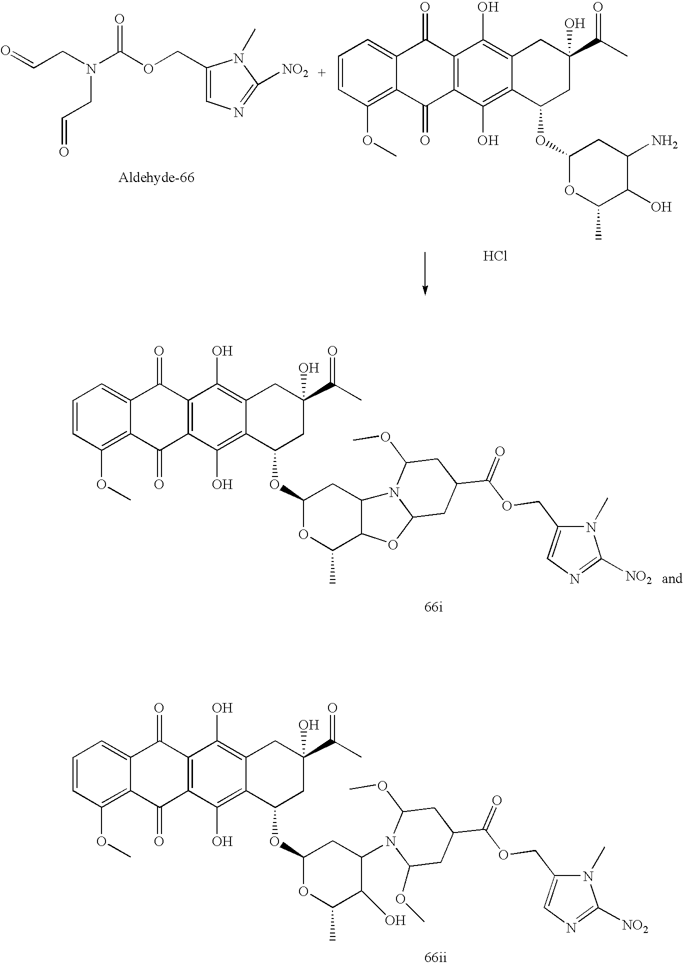

A mixture of Aledyde-66 (20 mg, 0.07 mmol), daunorubicin (1 eq, 40 mg, 0.07 mmol), and Methanol (2 mL) was stirred at room temperature overnight to yield Compound 66 as a mixture of 66i and 66ii.
Synthesis of Compounds 67 and 68.

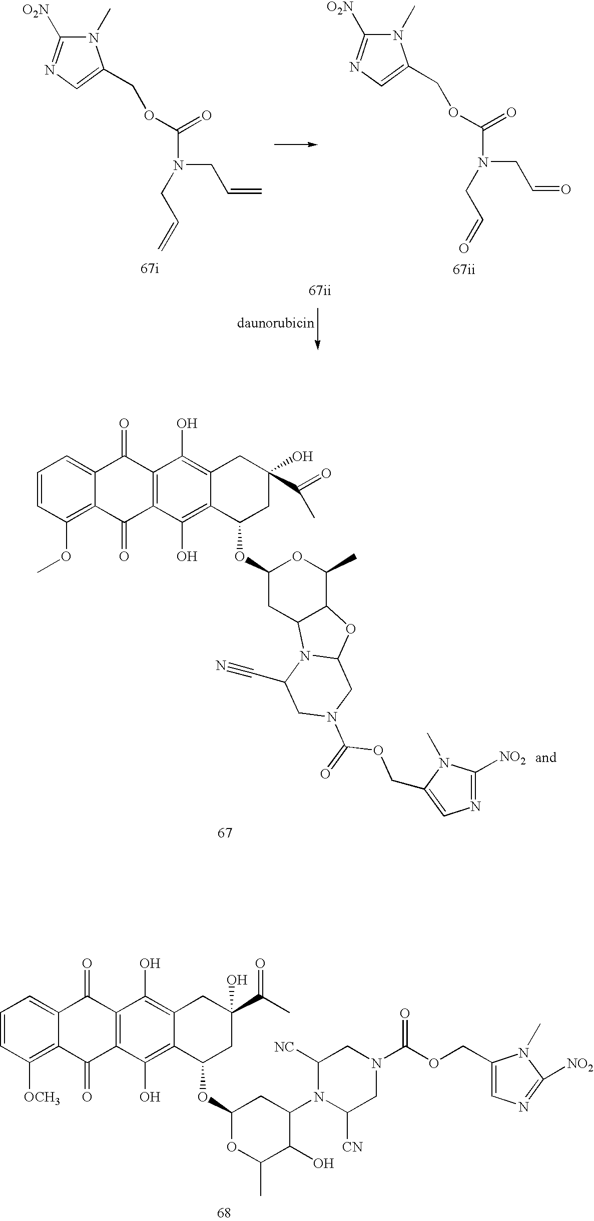

A mixture of 67i (1.330 g, 4.67 mmol), ether (25 mL), water (25 mL), and Osmium tetroxide (0.1 eq, 118.9 mg) was stirred while sodium periodate (5 eq, 5.00 g) was added in a 30 minutes period. The temperature was maintained at 25° C. during the addition and the reaction mixture stirred at room temperature overnight. The reaction mixture was diluted with Ethyl acetate, the organic layer separated, washed 3 times with water, and concentrated to yield a residue which was separated by column chromatography to yield Compound 67ii. A mixture of daunorubicin (56.5 mg, 0.15 mmol), DCM (4 mL), Aldehyde-67ii (1.5 eq, 42.6 mg) was stirred for 30 minutes, the reaction mixture diluted with 5 mL acetonitrile, and a solution of KCN (10 eq, 65.1 mg) in 1 mL water added to it (which is adjusted to pH 6-7 by addition of acetic acid). After stirring for 1 hour, the reaction mixture was diluted with 5% aqueous sodium bicarbonate solution, extracted with DCM, the DCM portion concentrated to yield a residue which was separated by column chromatography to yield compounds 67 and 68.
Synthesis of Compound 5

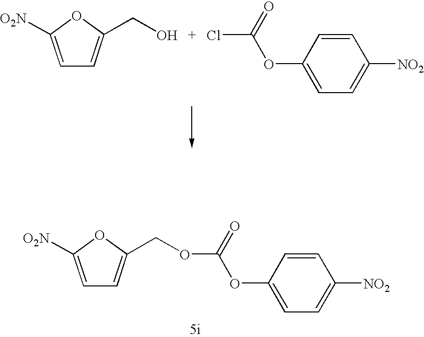

To 5-Nitrofurfuryl alcohol (3.5 g, 24.45 mmol) in 90 mL THF was added a solution of 4-nitrophenyl chloroformate (1 eq, 4.93 g) in 50 mL THF and stirred at room temperature overnight. The reaction mixture was diluted with 200 mL DCM, washed 2 times with 1% HCl and 2 times with aqueous sodium bicarbonate solution, the organic portion was concentrated to yield a residue which was separated by column chromatography to yield Compound 5i.

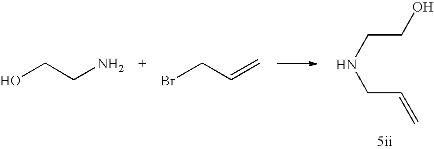

Allyl bromide (5.0 g, 41.32 mmol) was added dropwise into vigorously stirred ethanolamine (2.5 eq, 6.31 g) at 60° C. After 2 hours, a solution of KOH (2 eq, 4.64 g) in water (4 mL) was added and stirred for 5 minutes. The reaction mixture was cooled down to room temperature and stirred overnight. The reaction mixture was filtered and the filtrate extracted with ether to yield Compound 5ii. 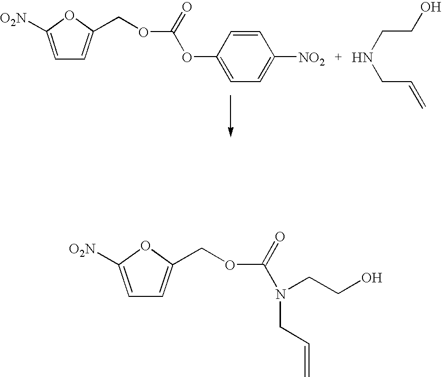
5iii

A mixture of Compound 5i (1.20 g, 3.89 mmol), 5ii (1.2 eq, 476.6 mg), and DIEA (3 eq, 2 mL) in DMF (20 mL), was stirred at room temperature overnight. The reaction mixture was then diluted with water, extracted 3 times with ethyl acetate, the organic portion concentrated to yield a residue which was separated by column chromatography to yield Compound 5iii.

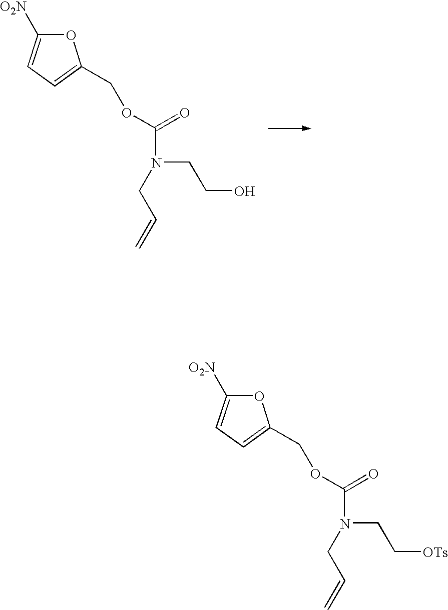

Compound 5iii (300 mg, 1.11 mmol) in 10 mL THF, was added a solution of p-toluenesulfonyl chloride (1.2 eq, 254 mg) in 5 mL THF at rt, the reaction mixture cooled down to 0° C., 1,8-Diazabicyclo [5.4.0]-undec-7-ene (1.2 eq, 202.5 mg) added to it, and stirred at rt overnight. The reaction mixture was diluted with water and extracted with ethyl acetate. The organic layer was concentrated to yield a residue which was separated by flash column chromatography (eluent: 10% to 80% of Ethyl acetate in Hexane) to yield Compound 5iv.

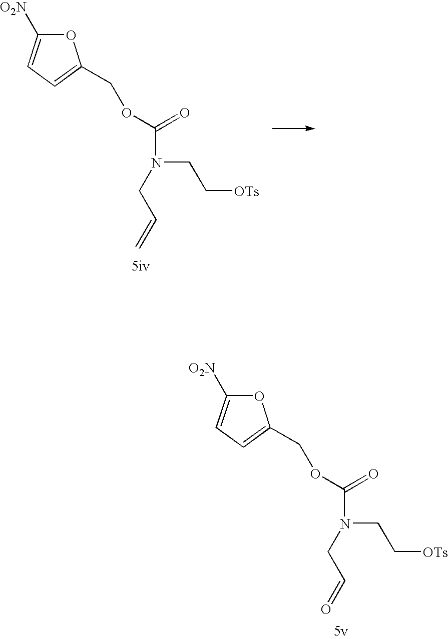

A mixture of Compound 5iv (450 mg, 1.06 mmol), ether (5 mL), water (5 mL), and osmium tetroxide (0.05 eq, 13.47 mg) was stirred while sodium periodate (2.5 eq, 566.8 mg) was added to it over a 30 minute period while temperature of the reaction mixture was maintained at 25° C. during the addition. The reaction mixture was stirred overnight at room temperature, extracted with Ethyl acetate, the organic layer separated, washed 3 times with water, and concentrated to yield a residue which was separated by flash column chromatography (eluent: 50% to 100% of Ethyl acetate in Hexane).

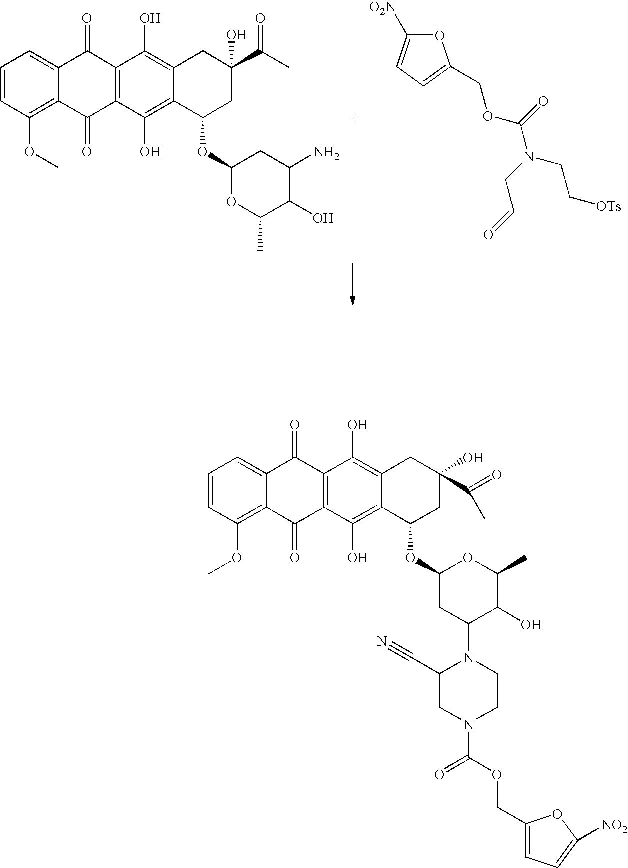

A mixture of daunorubicin (106.1 mg, 0.187 mmol) and potassium Cyanide (1 eg, 12.2 mg) in 2 mL Methanol, was stirred at room temperature for 30 minutes followed by the addition of 5v (1 eq, 80 mg) in 2 mL Methanol and the reaction mixture stirred overnight. The reaction mixture was concentrated to yield a residue which was separated by flash column chromatography (eluent MeOH/DCM 2:98) to yield Compound 5.
Synthesis of Compound 28

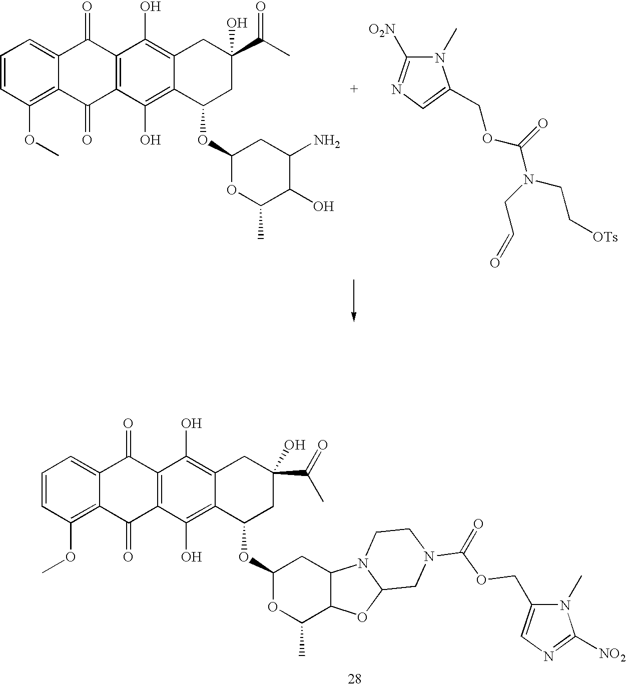

To a mixture of daunorubicin (61.6 mg, 0.109 mmol) and Aldehyde-28 (1 eq, 48 mg) in DMF (3 mL) was added DIEA (2 eq, 0.04 mL) and the reaction mixture stirred at rt overnight. The reaction mixture was diluted with water, extracted with DCM, the organic layer washed with sodium bicarbonate solution and concentrated to yield a residue which was separated by preparative TLC (eluent MeOH/DCM 10:90) to yield Compound 28.

Synthesis of Compound 69.

Aldehyde-69 was synthesized the same way as 5v, substituting

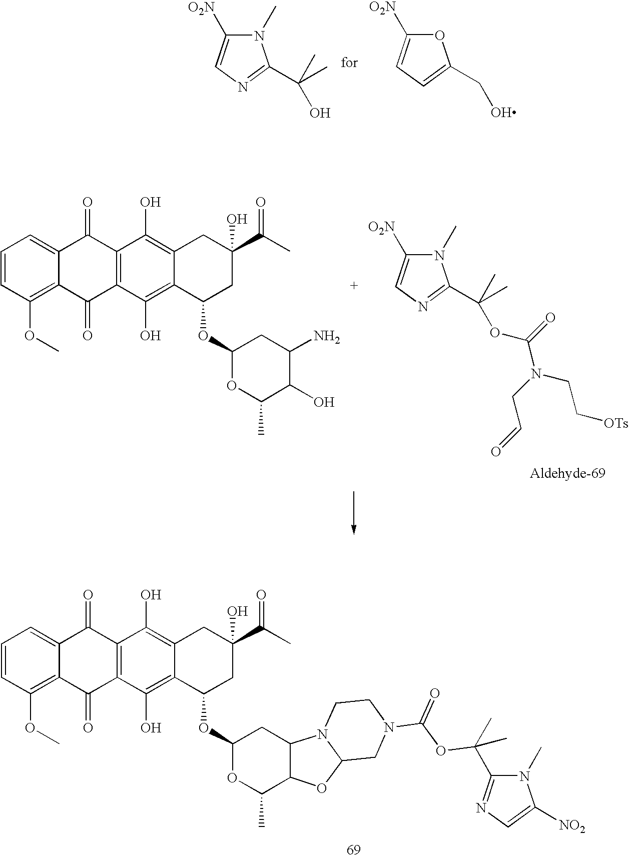

To a mixture of daunorubicin (4.22 mg, 0.0074 mmol) in 0.5 mL DMF was added Aldehyde-69 (1 eq, 3.5 mg) and DIEA (2 eq, 0.003 mL) and the reaction mxture stirred at room temperature overnight. The reaction mixture was diluted with DCM, the organic layer separated, washed with water, sodium bicarbonate solution, and concentrated to yield a residue which was separated by preparative TLC to yield Compound 69.
Synthesis of Compound 70.

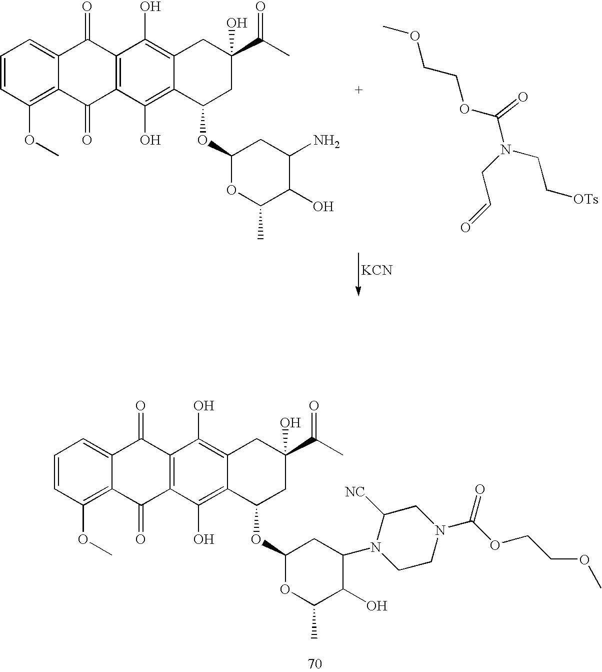

To daunorubicin (20 mg, 0.035 mmol) in 1 mL methanol/water (80:20), added KCN (1 eq, 2.3 mg), stirred for 30 minutes followed by the addition of Aldehyde-70 (1 eq, 12.2 mg) in 1 mL methanol/water (80:20) and stirred at room temperature overnight. Thereaction mixture was concentrated to yield a residue which was separated by column chromatography to yield Compound 70.
Synthesis of Compound 29i

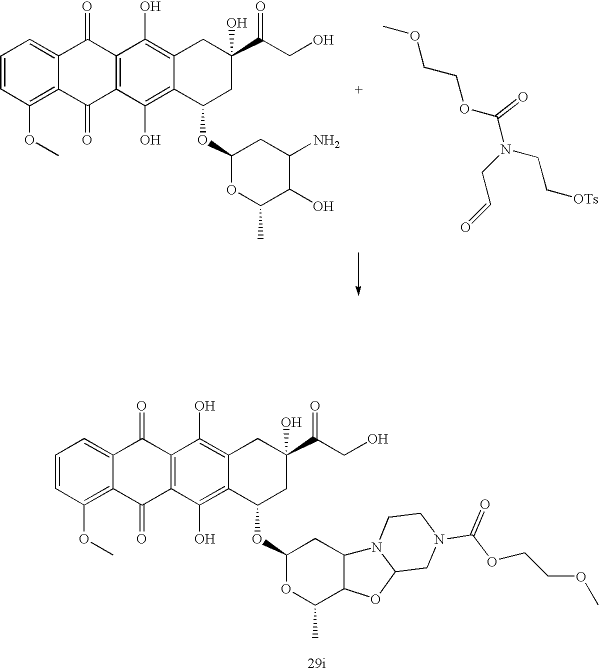

To a mixture of doxorubicin (20 mg, 0.034 mmol) and DMF (1 mL), was added Aldehyde-70 (1 eq, 12.4 mg) and DIFA (2 eq, 0.012 mmol), the reaction mixture was stirred overnight, diluted with DCM, the organic layer separated, washed with water and aqueous sodium bicarbonate solution, and concentrated to yield a residue which was separated by column chromatography to yield Compound 29i.
Synthesis of Compound 27.

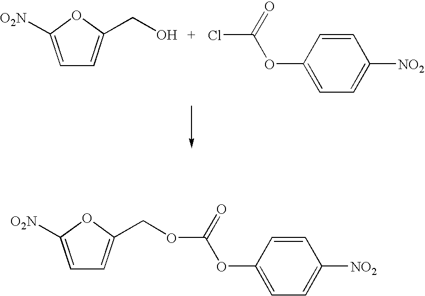

To 5-Nitrofurfuryl alcohol (3.500 g, 24.45 mmol) in 90 mL THF was added a solution of 4-nitrophenyl chloroformate (1 eq, 4.9 g) in 50 mL THF and stirred at rt overnight. The reaction mixture was diluted with 200 mL DCM, the organic layer separated, washed 2 times with 1% HCl and 2 times with sodium bicarbonate, concentrated to yield a residue which was separated by column chromatography to yield 5i.

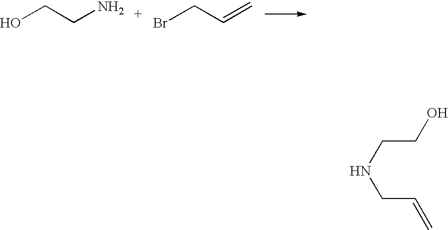

Allyl bromide (5.0 g, 41.32 mmol) was added dropwise into vigorously stirred ethanolamine (2.5 eq, 6.3 g) at 60° C. After 2 hours, a solution of KOH (2 eq, 4.6 g) in water (4 mL) was added and stirred for 5 minutes following which the reaction mixture was cooled down to rt and stirred overnight. the inorganic salt was filtered off, The reaction mixture was extracted with ether and the ether layer concentrated to yield Compound 5ii.

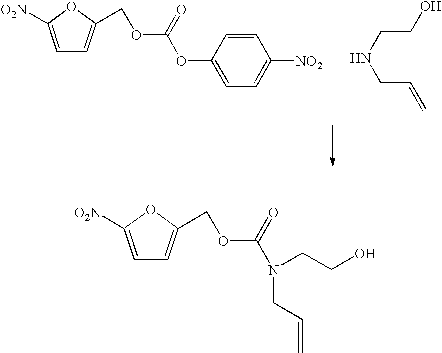

To 5i (1.2 g, 3.89 mmol) in DMF (20 mL) was added 5ii (1.2 eq, 476.6 mg), and DIEA (3 eq, 2 mL), stirring at rt overnight. The reaction mixture was diluted with water and extracted with ethyl acetate 3 times. The organic portion was concentrated and the residue separated by column chromatography to yield compound 5iii.

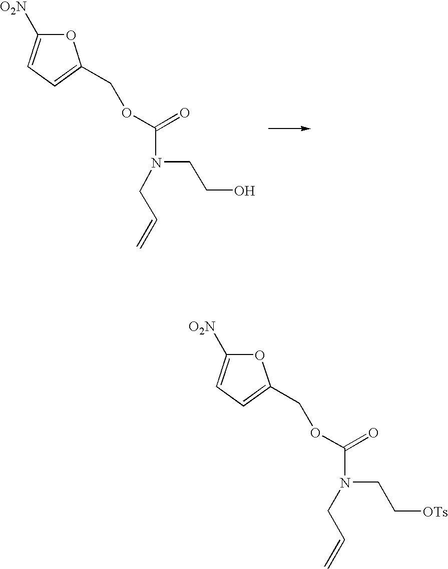

Compound 5iii (300 mg, 1.11 mmol) in 10 mL THF was added to a solution of p-toluenesulfunyl chloride (1.2 eq, 254 mg) in 5 mL THF at rt. The reaction mixture was cooled down to 0° C., 1,8-Diazabicyclo [5.4.0]-undec-7-ene (1.2 eq, 202.5 mg) added to I, and stirred at rt overnight. The reaction mixture was diluted with water and extractd with ethyl acetate. The organic portion was concentrated to yield a residue which was separated by column chromatography to yield compound 5iv.

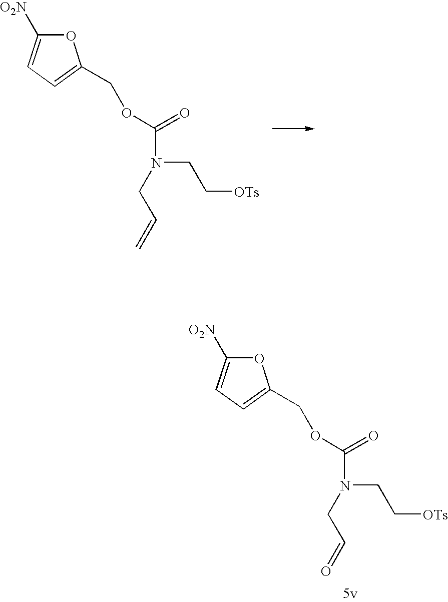

A mixture of Compound 5iv (450 mg, 1.06 mmol), ether (5 mL), water (5 mL), and Osmium tetroxide (0.05 eq, 13.47 mg) was stirred while sodium periodate (2.5 eq, 566.8 mg) was added over a 30 minutes period. The temperature was maintained at 25° C. during the addition and stirring continued at rt overnight. The reaction mixture was diluted with ethyl acetate, washed 3 times with water, and concentrated to yield a residue which was separated by column chromatography to compound 5v.

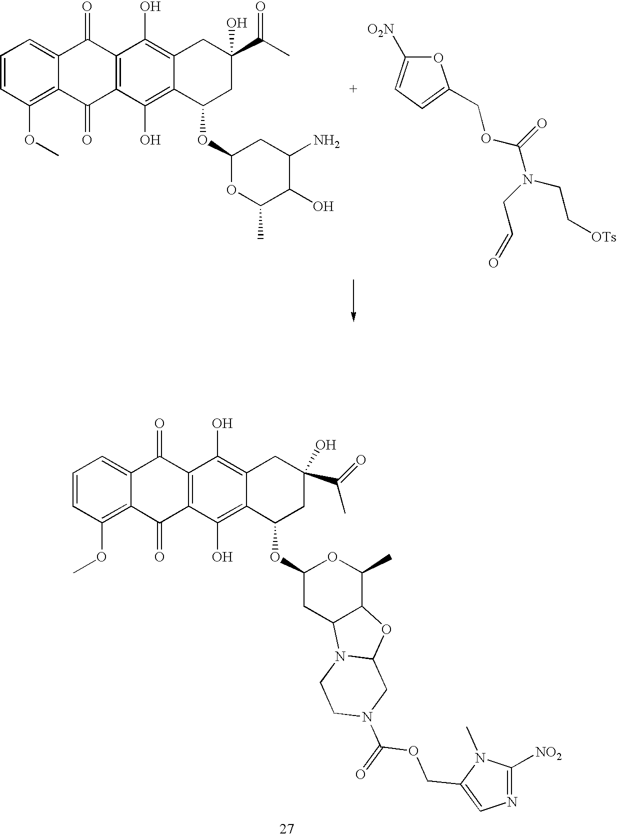

To a mixture of daunorubicin (26.5 mg, 0.04 mmol) in 1 mL DMF, was added 5v (1 eq, 20 mg), DIEA (2 eq), and stirred overnight. The reaction mixture was dilutd with water, extracted with DCM, the organic layer washed with aqueous sodium bicarbonate, concentrated to yield a residue which was separated by column chromatography to yield Compound 27.
Synthesis of Compound 71

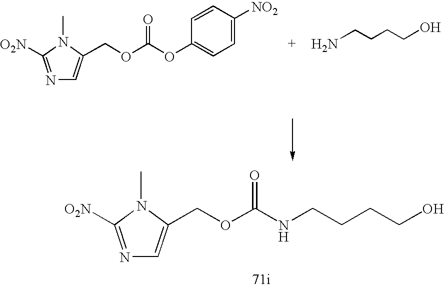

A mixture of 71i (200 mg), THF (5 mL), 4-Amino-1-butanol (1 eq, 55.4 mg), and DIEA (3 eq, 0.32 mL) was stirred overnight. Volatiles were removed in a rotary evaporator and the residue was separated by column chromatography, (eluent 1% to 20% of methanol in ethyl acetate).

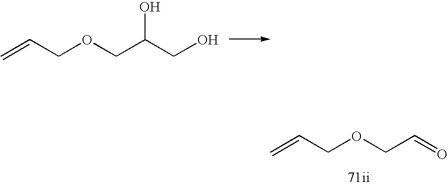

A mixture 3-Allyloxy-1,2-propanediol (1.0 g), ether (10 ml), and NaIO4 (in 10 mL of water) was stirred at rt for 2 hours. The organic portion was separated, concentrated and the residue separated by column chromatography (eluent: 0% to 20% of methanol in DCM).

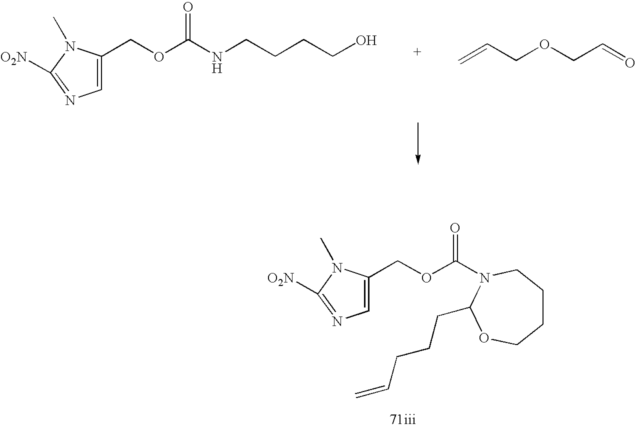

A mixture of Compound 71i (100 mg), 71ii (2.5 eq, 55.4 mg), and TsOH (0.1 eq, 7 mg) in THF (2 mL) was stirred and refluxed for 3 hours. The reaction mixture was concentrated and the residue separated by column chromatography to yield Compound 71 iii.

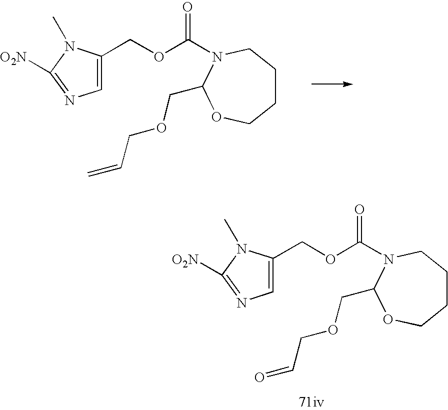

A mixture of Compound 71 iii (30.8 mg), Ether(0.5 mL), Water(0.5 mL), and OsO4(1.1 mg) were stirred while NaIO4 was added over a 30 minutes period, and the temperature was maintained at 25° C. during the addition. After stirring the reaction mixture at rt for 2 hours, it was extracted with Ethyl acetate. The organic portion was concentrated and the residue was separated by column chromatography (eluent: 0% to 50% of methanol in ethyl acetate) to yield Compound 71 iv.

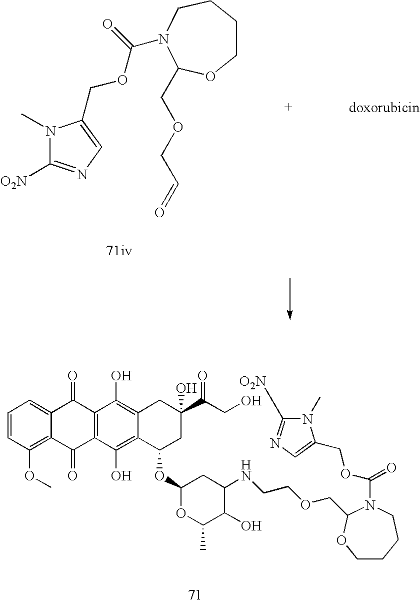

A mixture of Compound 71iv (30 mg) and Doxorubicin (1 eq, 49 mg) in DCM (2 ml) was stirred at rt for 30 minutes, the reaction mixture cooled to 0° C., NaBH(OAc)₃ (2 eq, 36 mg) added to it, and stirred for 30 minutes. The reaction mixture was separated by column chromatography (eluent: 5% to 15% of methanol in DCM).

B. Cytotoxicity of anthracycline analogs

Cytotoxicity of the compounds of the present invention were measured by their anti-proliferation activity on H460 cell following 2 h and/or 3 day incubation and the results, described as a concentration of the compound required to inhibit cellular proliferation by 50%, GI₅₀, are provided in Table 1 below. Cell growth in the presence and absence of the test compound was compared following a multi-well Alamar Blue based assay, and measured by a fluorescence plate reader at 550 nm excitation and 590 nm emission (see Biosource International Inc., Tech Application Notes, Use of Alamar Blue in the measurement of Cell Viability and Toxicity, Determining IC₅₀). H460 cells (ATCC HTB-177 (NCl—H40), 4,000 cells/well/200 μl) were seeded in a 96 well plate in RPMI medium (Invitrogen Corporation, Carlsbad, Calif.). After 24 hours, these plates were divided into 3 groups—Control group, 2 h treatment group and 3 day treatment group. A test compound was added in a range of concentration to each plate in the treatment groups (2 h and 3 day). In the 2 h treatment group, after 2 h, the H460 cells were rinsed to remove the test compound, incubated for 3 days, and stained with AlamarBlue. The cells in the 3-day treatment group were incubated along with the test compound for 3 days, followed by staining with AlamarBlue. In the Control group, AlamarBlue was added to the plate at (i) day 0 and (ii) day 3 and the fluorescence emission measured to establish the control reading. In all the groups, the capacity of the cells to proliferate was measured 6 hours after addition of AlamarBlue by a fluorescence plate reader at 550 nm excitation and 590 nm emission.

TABLE 1

| | | |
|---|---|---|
| Compound | GI₅₀ (nM) | GI₉₀ (nM) |

| No. | 2 h | 3 day | 2 h | 3 day |
|---|---|---|---|---|
| 1 | 25.00 | 15.80 | 125.90 | 79.40 |
| 3 | 2.50 | 1.30 | 7.90 | 4.00 |
| 5 | 3.20 | 1.00 | 10.00 | 4.00 |
| 13 | 10.00 | 4.0 | 50.10 | 20.00 |
| 14 | 0.20 | 0.20 | 0.40 | 0.63 |
| 27 | 0.60 | 0.80 | 1.60 | 1.60 |
| 28 | 0.03 | 0.40 | 0.100 | 1.258 |
| 29 | 0.04 | 0.40 | 0.16 | 1.26 |
| 30 | 0.04 | 0.16 | 0.16 | 0.79 |
| 31 | 2.50 | 0.60 | 15.80 | 5.00 |
| 32 | 10.00 | 4.00 | 50.10 | 20.00 |
| 33 | 20.00 | 10.00 | 79 | 50 |
| 43 | 63.10 | 12.60 | 199.50 | 63.00 |
| 44 | 1000 | 100 | — | 501 |
| 45 | 630 | 158 | 1000 | 630 |
| 46 | 50.10 | 20.00 | 158.50 | 100.00 |
| 47 | 125.90 | 20.00 | 398.1 | 100.00 |
| 48 | 141.30 | 63.10 | 398.10 | 199.50 |
| 49 | 63.10 | 20.00 | 158.50 | 100.00 |
| 50 | 0.13 | 0.16 | 0.40 | 0.32 |
| 51 | 2.5 | | | |
| 52 | 0.3 | | | |
| 53 | 15.80 | 2.50 | | |
| 54 | 3.98 | 1.00 | 25.10 | 3.20 |
| 55 | 3.98 | 0.20 | 25.00 | 1.6 |
| 56 | 31.60 | 39.80 | 398 | 158.5 |
| 57 | 125 | 1.58 | 316 | 10 |
| 58 | 0.30 | 0.14 | 1.00 | 0.60 |
| 59 | 199.5 | 50 | 1000 | 199.5 |
| 60 | 0.79 | 0.10 | 2.50 | 0.20 |
| 61 | 15.80 | 79.40 | 158.40 | 398.10 |
| 62 | 0.25 | 0.10 | 1.30 | 0.25 |
| 63 | 3.98 | 0.80 | 12.60 | 3.20 |

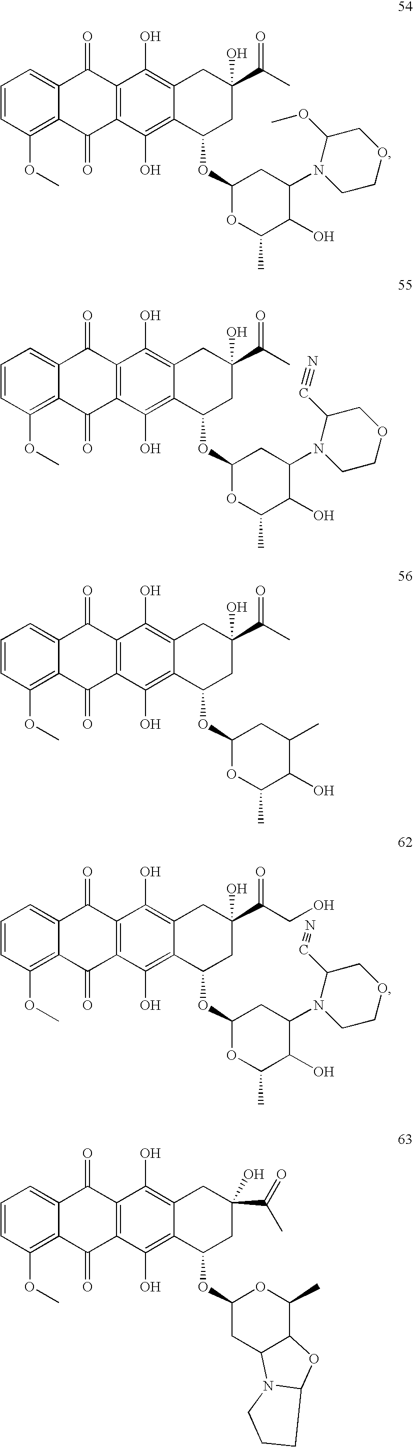

Although the present invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

We claim:

1. A compound having structure of Formula (I) 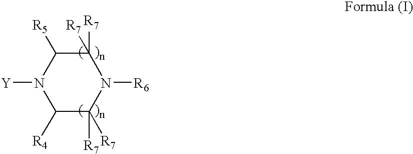
wherein each n is independently 1-3;
Y is selected from the group consisting of: 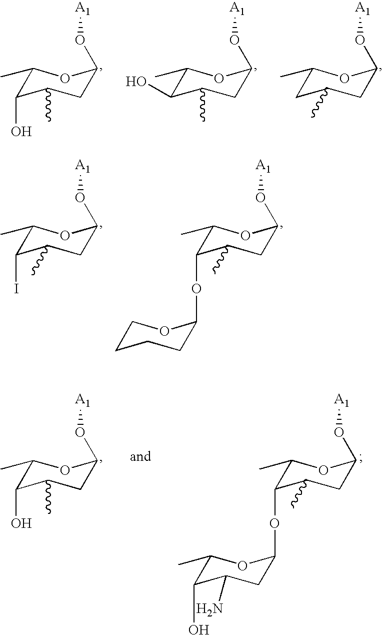
wherein A₁ is 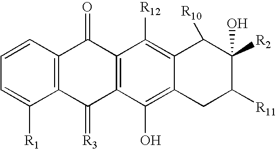
wherein
R₁ is hydrogen, substituted or unsubstituted C₁-C₆ alkyl or heteroalkyl, hydroxyl, C₁-C₆ alkoxy, amino, C₁-C₆alkylamino, C₁-C₆ dialkylamino, mercapto, and C₁-C₆ alkylthio;
R₂ is selected from the group consisting of —CH₂CH₃, —COCH₃, —CH(OH)CH₃, —COCH₂OH, —CH(OH)CH₂OH, —C(═N-Z₁)-CH₃, and —C(═N-Z₁)-CH₂OH wherein Z₁ is —OZ₂ or —N(Z₂)₂ wherein each Z₂ is selected from the group consisting of hydrogen, substituted or unsubstituted C₁-C₆ alkyl or heteroalkyl, and substituted or unsubstituted C₁-C₆ aryl or heteroaryl;
R₃ is O or NH;
R₁₀ and R₁₁ each independently is hydrogen, hydroxyl, or halogen;
R₁₂ is hydrogen or hydroxyl;
each n is independently 1-3;
R₄ and R₅ are each independently hydrogen, hydroxyl, C₁-C₆ alkoxy, cyano, amino, C₁-C₆ alkylamino, C₁-C₆ dialkylamino, mercapto, or C₁-C₆ alkylthio;
R₆ is —(CO₂)ₙ-Z₃ or —(CO)-Z₃ wherein n is 0 or 1 and Z₃ is selected from the group consisting of hydrogen, substituted or unsubstituted C₁-C₆ alkyl or heteroalkyl, substituted or unsubstituted C₁-C₆ aryl or heteroaryl, and —C(Z₄)₂(CZ₄═CZ₄)₂Z₃ wherein each Z₄ is independently hydrogen,halogen, substituted or unsubstituted C₁-C₆ alkyl or heteroalkyl, substituted or unsubstituted C₁-C₆ aryl or heteroaryl, C₁-C₆ acyl or Cl-C₆ heteroacyl, aroyl, and heteroaroyl with the proviso that when n is 0 then Z₃ is not hydrogen;
each R₇ independently is hydrogen, substituted or unsubstituted C₁-C₆ alkyl or heteroalkyl, substituted or unsubstituted C₁-C₆ aryl or heteroaryl; and
an individual isomer or a racemic or non-racemic mixture of isomers, a pharmaceutically acceptable salt, solvate, hydrate, or a prodrug thereof.

2. The compound of claim 1 wherein Y is selected from the group consisting of:

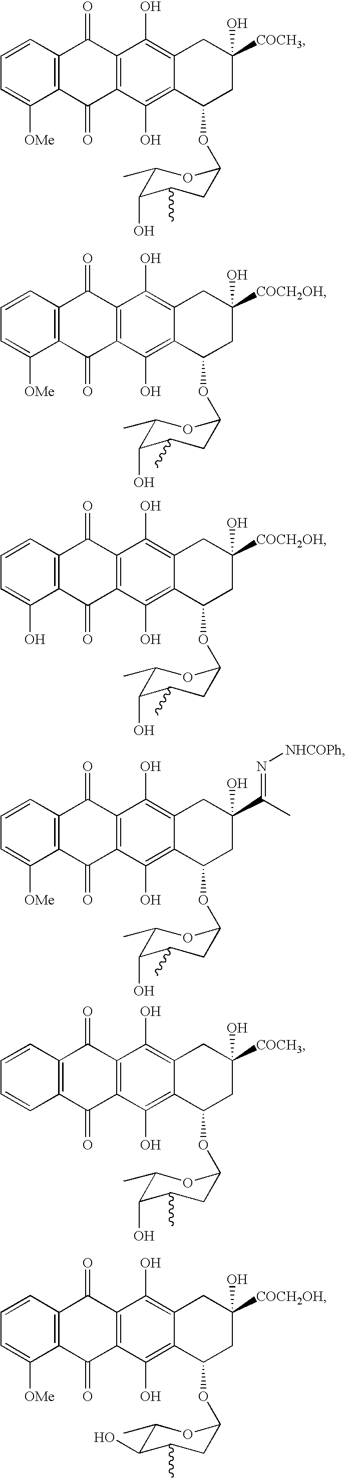

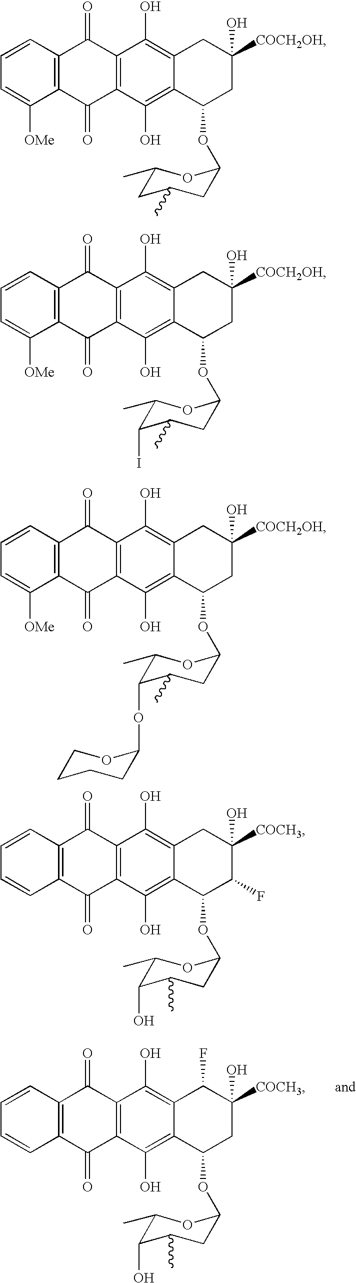

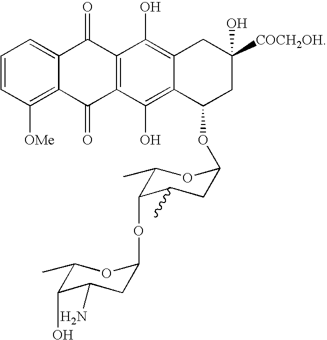

3. A compound having structure of Formula (II): 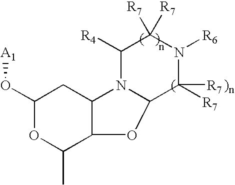
wherein A₁ is 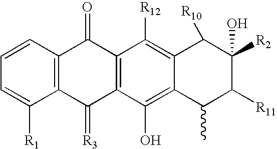
wherein
R₁ is hydrogen, substituted or unsubstituted C₁-C₆ alkyl or heteroalkyl, hydroxyl, C₁-C₆ alkoxy, amino, C₁-C₆alkylamino, C₁-C₆ dialkylamino, mercapto, and C₁-C₆ alkylthio;
R₂ is selected from the group consisting of —CH₂CH₃, —COCH₃, —CH(OH)CH₃, —COCH₂OH, —CH(OH)CH₂OH, —C(═N-Z₁)-CH₃, and —C(═N-Z₁)-CH₂OH wherein Z₁ is —OZ₂ or —N(Z₂)₂ wherein each Z₂ is selected from the group consisting of hydrogen, C₁-C₆ alkyl or heteroalkyl, C₃-C₈ cycloalkyl or heterocyclyl, and aryl or heteroaryl;
R₃ is O or NH;
R₁₀ and R₁₁ each independently is hydrogen, hydroxyl, or halogen;
R₁₂ is hydrogen or hydroxyl;
each n is independently 1-3;
R₄ and R₅ each independently is hydrogen, hydroxyl, C₁-C₆ alkoxy, cyano, amino, C₁-C₆ alkylamino, C₁-C₆ dialkylamino, mercapto, or C₁-C₆ alkylthio;
R₆ is —(CO₂)ₙ-Z₃ or —(CO)-Z₃ wherein n is 0 or 1 and Z₃ is selected from the group consisting of hydrogen, C₁-C₆ alkyl or heteroalkyl, C₃-C₈ cycloalkyl or heterocyclyl, aryl or heteroaryl, C₁-C₆ alkylamino or di C₁-C₆ alkylamino and —C(Z₄)₂(CZ═CZ₄)₂Z₃ wherein each Z₄ is independently hydrogen, halogen, substituted or unsubstituted C₁-C₆ alkyl or heteroalkyl, substituted or unsubstituted C₁-C₆ aryl or heteroaryl, C₁-C₆ acyl or C₁-C₆ heteroacyl, aroyl, and heteroaroyl with the proviso that when n is 0 then Z₃ is not hydrogen; and
each R₇ independently is hydrogen, substituted or unsubstituted C₁-C₆ alkyl or heteroalkyl, substituted or unsubstituted C₁-C₆ aryl or heteroaryl; and
an individual isomer or a racemic or non-racemic mixture of isomers, a pharmaceutically acceptable salt, solvate, hydrate, or a prodrug thereof.

4. The compound of claim 3 wherein A₁ is selected from the group consisting of

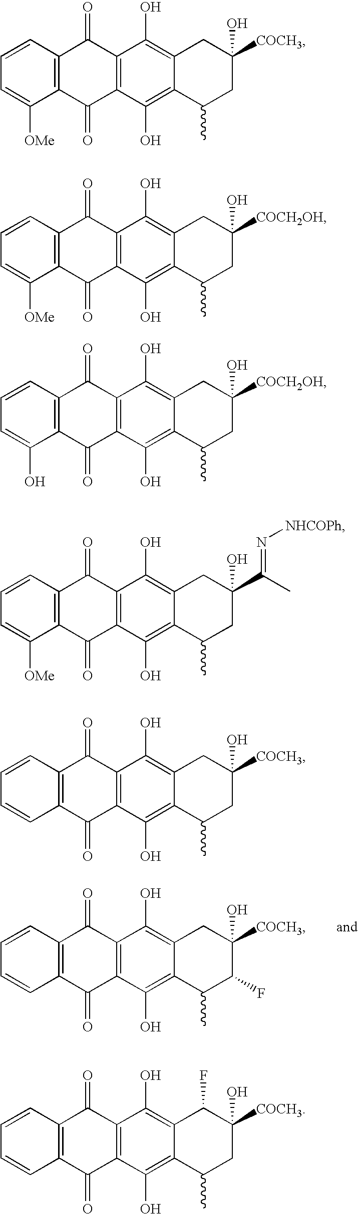

5. A compound having structure of Formula (III) 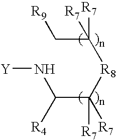
wherein Y is selected from the group consisting of: 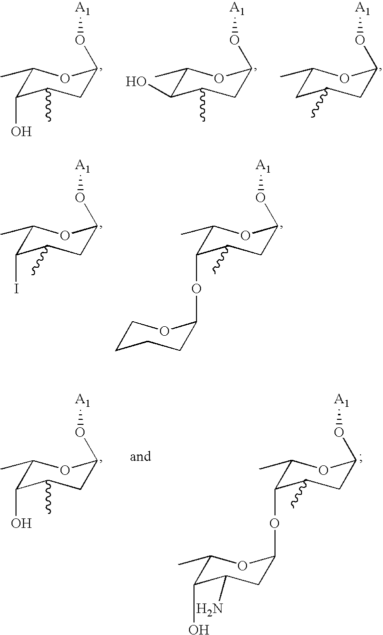
wherein A₁ is 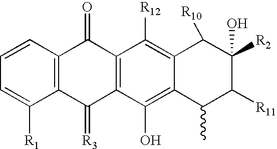
wherein
R₁ is hydrogen, substituted or unsubstituted C₁-C₆ alkyl or heteroalkyl, hydroxyl, C₁-C₆ alkoxy, amino, C₁-C₆ alkylamino, C₁-C₆ dialkylamino, mercapto, and C₁-C₆ alkylthio;
R₂ is selected from the group consisting of —CH₂CH₃, —COCH₃, —CH(OH)CH₃, —COCH₂OH, —CH(OH)CH₂OH, —C(═N-Z,)-CH₃, and —C(═N-Z,)-CH₂OH wherein Z₁ is —OZ₂ or —N(Z₂)₂ wherein each Z₂ is selected from the group consisting of hydrogen, C₁-C₆ alkyl or heteroalkyl, C₃-C₈ cycloalkyl or heterocyclyl, and aryl or heteroaryl;
R₃ is O or NH;
R₁₀ and R₁₁ each independently is hydrogen, hydroxyl, or halogen;
R₁₂ is hydrogen or hydroxyl;
each n is independently 1-3;
R₄ and R₅ each independently is hydrogen, hydroxyl, C₁-C₆ alkoxy, cyano, amino, C₁-C₆ alkylamino, C₁-C₆ dialkylamino, mercapto, or C₁-C₆ alkylthio;
R₆ is —(CO₂)ₙ-Z₃ or —(CO)-Z₃ wherein n is 0 or 1 and Z₃ is selected from the group consisting of hydrogen, C₁-C₆ alkyl or heteroalkyl, C₃-C₈ cycloalkyl or heterocyclyl, aryl or heteroaryl, C₁-C₆ alkylamino or di C₁-C₆ alkylamino and —C(Z₄)₂(CZ₄═CZ₄)₂Z₃ wherein each Z₄ is independently hydrogen,halogen, substituted or unsubstituted C₁-C₆ alkyl or heteroalkyl, substituted or unsubstituted C₁-C₆ aryl or heteroaryl, C₁-C₆ acyl or C₁-C₆ heteroacyl, aroyl, and heteroaroyl with the proviso that when n is 0 then Z₃ is not hydrogen; each R₇ independently is hydrogen, substituted or unsubstituted C₁-C₆ alkyl or heteroalkyl, substituted or unsubstituted C₁-C₆ aryl or heteroaryl; R₈ is O,S, NR₆, or C(R₆)₂ wherein R₆ is defined as above; and
R₉ is halo, alkylsufonyloxy, heteroalkylsufonyloxy, arylsulfonyloxy, and heteroalkylsulfonyloxy; and
an individual isomer or a racemic or non-racemic mixture of isomers, a pharmaceutically acceptable salt, solvate, hydrate, or a prodrug thereof.

6. The compound of claim 5 wherein Y is selected from the group consisting of:

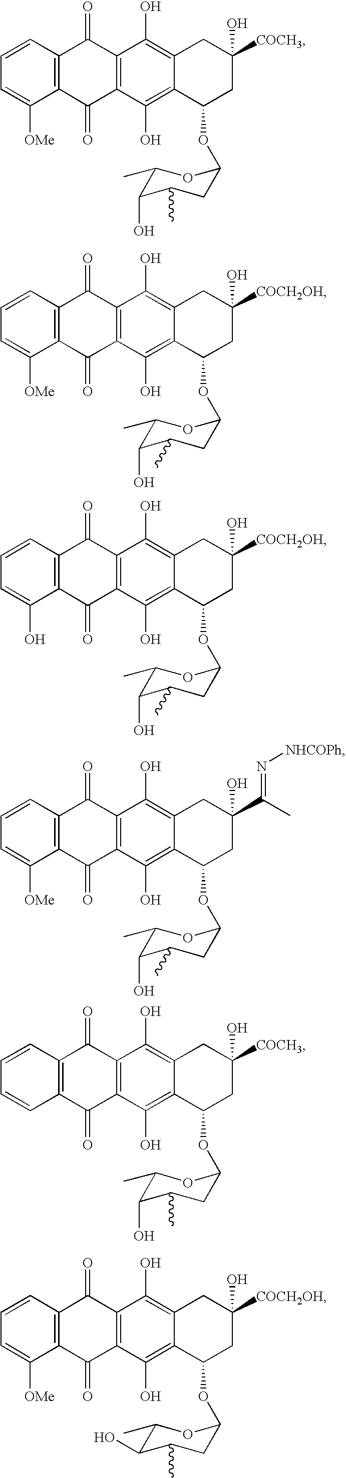

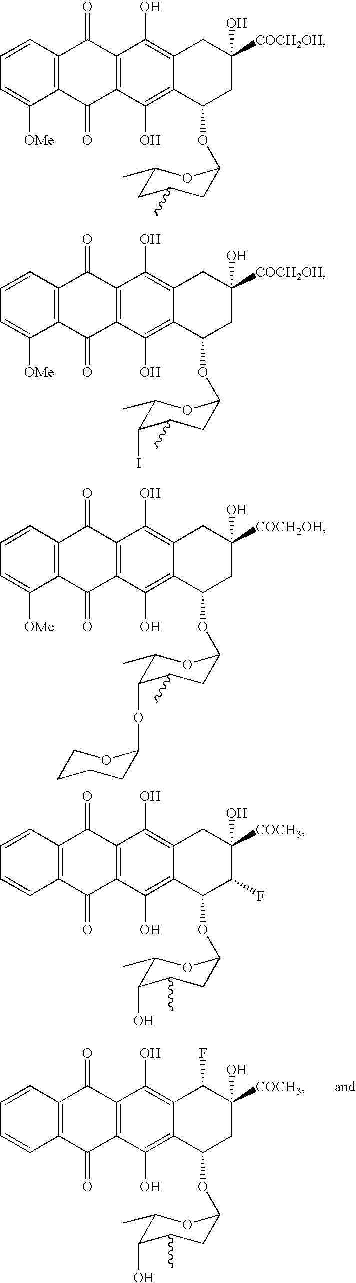

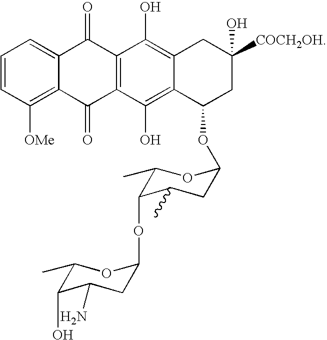

7. A method of making a compound comprising reacting:
(i) Y—NH₂ wherein
Y is selected from the group consisting of: 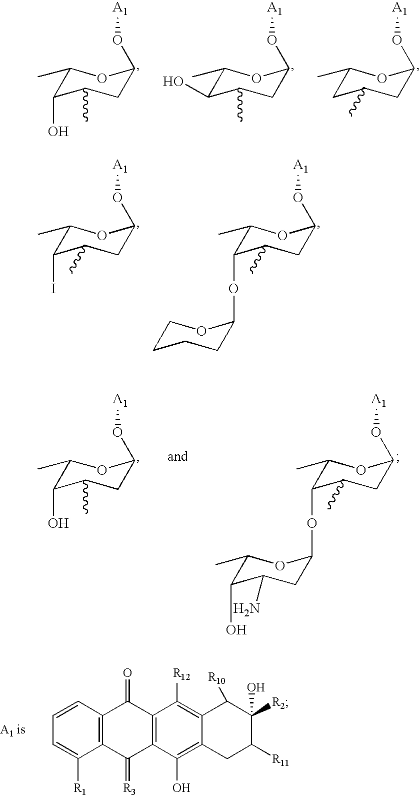
R₁ is hydrogen, substituted or unsubstituted C₁-C₆ alkyl or heteroalkyl, hydroxyl, C₁-C₆ alkoxy, amino, C₁-C₆alkylamino, C₁-C₆ dialkylamino, mercapto, and C₁-C₆ alkylthio;
R₂ is selected from the group consisting of —CH₂CH₃, —COCH₃, —CH(OH)CH₃, —COCH₂OH, —CH(OH)CH₂OH, —C(═N-Z₁)-CH₃, and —C(═N-Z₁)-CH₂OH wherein Z, is —OZ₂ or —N(Z₂)₂ wherein each Z₂ is selected from the group consisting of hydrogen, C₁-C₆ alkyl or heteroalkyl, C₃-C₈ cycloalkyl or heterocyclyl, and aryl or heteroaryl;
R₃ is O or NH;
R₁₀ and R₁₁ each independently is hydrogen, hydroxyl, or halogen;
R₁₂ is hydrogen or hydroxyl; and
each n is independently 1-3;
(ii) a compound having structure of Formula (VII) ot (VIII) 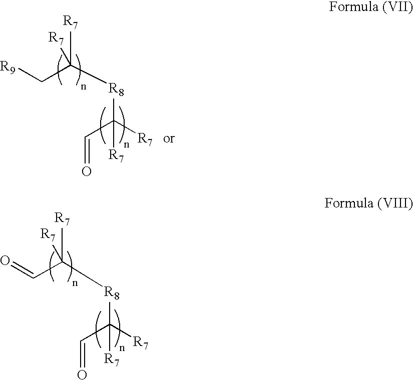
wherein each n is independently 1-3; L is a leaving group; R₆ is —(CO₂)ₙ-Z₃ or —(CO)-Z₃ wherein n is 0 or 1 and Z₃ is selected from the group consisting of hydrogen, substituted or unsubstituted C₁-C₆ alkyl or heteroalkyl, substituted or unsubstituted C₁-C₆ aryl or heteroaryl, and —C(Z₄)₂(CZ₄═CZ₄)₂Z₃ wherein each Z₄ is independently hydrogen,halogen, substituted or unsubstituted C₁-C₆ alkyl or heteroalkyl, substituted or unsubstituted C₁-C₆ aryl or heteroaryl, C₁-C₆ acyl or C₁-C₆ heteroacyl, aroyl, and heteroaroyl with the proviso that when n is 0 then Z₃ is not hydrogen; each R₇ independently is hydrogen, substituted or unsubstituted C₁-C₆ alkyl or heteroalkyl, substituted or unsubstituted C₁-C₆ aryl or heteroaryl; and R₈ is O, S, NR₆, or C(R₆)₂and
(iii) optionally a cyanide salt; to yield the compound.

8. The method of claim 7 wherein said reacting is carried out in the presence of a reducing agent.

9. A pharmaceutical composition comprising the compound of claim 1 and a pharmaceutically acceptable carrier or diluent.

10. A pharmaceutical composition comprising the compound of claim 2 and a pharmaceutically acceptable carrier or diluent.

11. A pharmaceutical composition comprising the compound of claim 3 and a pharmaceutically acceptable carrier or diluent.

12. A pharmaceutical composition comprising the compound of claim 4 and a pharmaceutically acceptable carrier or diluent.

13. A pharmaceutical composition comprising the compound of claim 5 and a pharmaceutically acceptable carrier or diluent.

14. A pharmaceutical composition comprising the compound of claim 6 and a pharmaceutically acceptable

15. A method of treating cancer comprising administering to a person in need of therapy thereof the compound of claim 1.

16. A method of treating cancer comprising administering to a person in need of therapy thereof the compound of claim 2.

17. A method of treating cancer comprising administering to a person in need of therapy thereof the compound of claim 3.

18. A method of treating cancer comprising administering to a person in need of therapy thereof the compound of claim 4.

19. A method of treating cancer comprising administering to a person in need of therapy thereof the compound of claim 5.

20. A method of treating cancer comprising administering to a person in need of therapy thereof the compound of claim 6.
